SEQUENCE LISTING (43 sequences)

SEQ ID NO: 1 - DNA, 300 bp, Escherichia coli
tgacggatggcctttttgcattggcgcagaaaaaaatgcctgatgcgacgctgcgcgtcttatactcccacatatgccagattcagcaacggatacggcttccccaacttgcccacttccatacgtgtcctccttaccagaaatttatccttaagctcctcaataaccattttcctgctaactaaattcatggttaaggttgcataatgatatgcaacaaatgtataatatttcctttacaaaaaaaataaacaaaagcgaccgacaaaagcatcggattacggcaggagacataatggc

SEQ ID NO: 2 - DNA, 300 bp, Escherichia coli
cgtcaagcaaaagtaaatcaatgctgtcgtccagaagatcaagcgcctgttccccatcgtgggcaacaatcacgttgaagccttccatctcgagcagctcctttaatagggaagtcagctctcggtcatcatcaactaacaggattttattcattgtttaaatacctccgaggcagaaattacgtcatcagacgtcgctaatccatgactttacgttgttttacaccccctgacgcatgtttgcagcctgaatcgtaaactctctatcgttgaatcgcgacagaaagattttgggagcaa

SEQ ID NO: 3 - DNA, 300 bp, Escherichia coli
aatgctcgaggaaatcttctctgtaaatttggcgtaaataatcagttacatcaatgagtcctaaacgaaatccatgtgtgaagttgatcacaaatttaaacactggtagggtaaaaaggtcattaactgcccaattcaggcgtcaactggtttgattgtacattccttaaccggagggtgtaagcaaacccgctacgcttgttacagagattgcatcctgcaattcccgctccccttttgcggccgtcgcgctgatttttctggcgtttgcggaaatgggccaactctgcgaggaaagct

SEQ ID NO: 4 - DNA, 300 bp, Escherichia coli
tgctgcgtcctgcattcaccagttgagtatcaagctttttgtccgccatgtcgggattcctgtttttatacgtctggatgtctaaactagcatgaatattcgcgggcgcatcctgaagaattggcataaagcagaaaattttcgcaaatggcgtgctggtgacttttttactgcgtaaacgcaaggaaaagaaagcaaaggtacggcagtatgcaaatagtaatgagaacgactatcaattcgacgtcgttttgatattattatgcgcagattttgtgacttgcgtcctggagatacaca

SEQ ID NO: 5 - DNA, 300 bp, Escherichia coli
ctttgttgatgcatgtcaaccacaaatctatcattcccccgatatatgtttattttatgtaaaatcaatttatgtaaaaagtcacatcattgtagttaaaaaggttgagttagatcgcagaaacgggtacatatagccccgcaaacgtgaccacgcccgcagatattacttaaatcagagccatagaggccacgcaggcgaggcatcaatctttacgatctgtataaagacggattgttgatgatgtgttaaaattgatgtaaacaaattgtgaagtgaatgtgcttccggggaaaataa

SEQ ID NO: 6 - DNA, 200 bp, Escherichia coli
ggcggactgctgccgtcaggtcaatatgctaaaaatcccttcatttctaataaccctataattaatcaacgaaattataatgtttctaaaattagaatataatttataaacattatttaaatgttgttacttaagtgttaccgttgatgccgcgcaaatctcacctgcaataaagcgactaaaagtaaggcattaacaag

SEQ ID NO: 7 - DNA, 300 bp, Escherichia coli
atgtgctgaacagcagtgaattgcatgcgccgctgcaaaagaatcaggtcgtcggaactatcaacttccagcttgatggcaaaacgatcgagcaacgcccgctggttgtgttgcaagaaatcccggaaggtaacttcttcggcaaaatcattgattacattaaattaatgttccatcactggtttggttaaaaattaaacacttgaaagtgtaatttccgtccccatatactaagcatcagtaaaaaactcccgccttctggcgggagttgctatttaattacgttacgccggagctgac

SEQ ID NO: 8 - DNA, 200 bp, Escherichia coli
accacctgcggaagaaaatcgcgaaatagcgcaacgagaacaagaaagttaacgctggttgattttccgaatttagcccttaaatcatcaacaatgcgtgtggatgccatttcgcagacggcgcgaaaatggtactttaaagggctattgcggtaagttgaccataatttattcgctctaaccacataacgggaagtaat

SEQ ID NO: 9 - DNA, 300 bp, Escherichia coli
cagctatgagcccggctaataaattcatgttcgccgggatgttgatgatgatgggagctggtttattgctttagttgtacgatgcaaaaaccaataaggaaacctgtgattttcagctctacatcaccctgcaaatctctgtcacttctaatataaaaatagggagaaatgatggagcttatattcattggcgattaggaaactatcttgttatacaaaacaatacagttctttacatttgccttgttttatgaatactcctgaagaggtgtataacataatggtacaagcagggtagat

SEQ ID NO: 10 - DNA, 300 bp, Escherichia coli
tagtttcgcgatcttcggtggcgattttcaccttgatgagttcatggtgctctaacgcttgttcaatctcggccagcaccccttcggtcaaaccattactgccaagcagaacaactggcttgagcggatgtgccagacctttcaggtgctgtttttgtttagtactcagattcatcgtattttttgcttacgttgggattgaaaacgggtcattctaccgccatctcccatatatcaccaaataggcgcgtaaaaatttacgcaattggttacgatgagttatccccatgggaaagttaa

SEQ ID NO: 11 - DNA, 300 bp, Escherichia coli
tctccgcgagcgtgccagttttcacattcttcagttgcagttcgtgagcgatttgttgctcaacgatgacctcgtaaccttttgtgcacagccagcggtagagcatttcatgtgttgtcagtgcagtggggtgccgtgggtgtcccacaatgccaatacacttgaaatgattattcatttttccgaggtccttgttgcgaagattgatgacaatgtgagtgcttcccttgaaaccctgaaactgatccccataataagcgaagttagcgagatgaatgcgaaaaaaacgcggagaaattc

SEQ ID NO: 12 - DNA, 300 bp, Escherichia coli
agattaccaggagccggatccttatctggatgagacggtgaatatcgcactcgatctggcgaagcttgaaaaagccagacccgcggaacaacccgctcccgtcaagtaatatcaatcaggcacaagaaattgtgcctgattttttaacagcgacaagatgccgtaaatcagatgctacaaaatgtaaagttgtgtctttctggtgacttacgcactatccagacttgaaaatagtcgcgtaacccatacgatgtgggtatcgcatattgcgttttgttaaactgaggtaaaaagaaaatt

SEQ ID NO: 13 - DNA, 300 bp, Escherichia coli
gataagtatctggcggatatttatcagcttgcccggcagcgtctggcgaacgtgggtgttgagcaaattttcggcggcgaccgttgtacatatacggaaaatgagactttcttctcttatcgtcgcgacaagaccaccggtcgtatggcaagtttcatttggctgatataacctaaagaatcaagacgatccggtacgcgtgattttcttttcacattaatctggtcaataaccttgaataattgagggatgacctcatttaatctccagtagcaactttgatccgttatgggaggagtt

SEQ ID NO: 14 - DNA, 200 bp, Escherichia coli
ccgtaatctggatcactttaagtgtcggtttttaccccttaattattaatttgtgaaatagatcaccgctttgggattactaccaaaaatagttgcgcaaacatcttgaaattttgctaatgaccacaatataagctaaacgcgattcgcaacccattcaggtagccggggttaaccggctgctattacaggagaaacct

SEQ ID NO: 15 - DNA, 200 bp, Escherichia coli
cgtgtggtcattggcccggtgaaaggcaaagagaacgcagacagcaccctcaatcggttgaagatggcgggtcatacaaactgcattcggctcgccgccgggggttgaaaccctcaaaatcccccccatctataattgcattatgccccgtacttttgtacggggtttgtactctgtattcgtaaccaaggggtcagctc

SEQ ID NO: 16 - DNA, 200 bp, Escherichia coli
ccaggtggtgggcttttttttgtcatgaattttgcatggaaccgtgcgaaaagcctctttcggtgttagcgtaacaacaaaagattgttatgcttgaaatatggtgatgccgtacccataacacagggactagctgataatccgtccataaggttacaatcggtacagcaggttttttcaattttatccaggagacggaa

SEQ ID NO: 17 - DNA, 200 bp, Escherichia coli
tcatggcgttccggttggcggcgagctggaaatggtcgacggcaccacgttgtcacactcccttgccgggcgtcataagattcgtttttaagcaaacgagagcaggatcacctgctctcgcttgaaattattctcccttgtccccatctctcccacatcctgtttttaaccttaaaatggcattattgaggtagacctac

SEQ ID NO: 18 - DNA, 200 bp, Escherichia coli
catatcgcgaaatttctgcgcaaaagcacaaaaaatttttgcatctcccccttgatgacgtggtttacgaccccatttagtagtcaaccgcagtgagtgagtctgcaaaaaaatgaaattgggcagttgaaaccagacgtttcgcccctattacagactcacaaccacatgatgaccgaatatatagtggagacgtttag

SEQ ID NO: 19 - DNA, 200 bp, Escherichia coli
gtattctcctgactttctcctgttccggtctgatgaccagcgatttatttcagaaaatcatcgcggatgccgcaattgatgccggtcgtgatgtacaatttatagagcagttccgtcaggcagccgatcatccggtgatcgctacctatccggaagggctatatctgaaagggtttgcctgtcgcgtcatgtaacttgaa

SEQ ID NO: 20 - DNA, 200 bp, Escherichia coli
tagcgccatttacaatggcgtacaggcctggcgtcgttaccagcgtcatcgcactcgcatgatggagattcaggcctattatgaaagctgcctgaacccgcaactgatcaccccttcagaaagccttatcgaataacacgtttgcgcggcaggttatgctaccctgtcgcgcaaattgcttcactctggagatttccctc

SEQ ID NO: 21 - DNA, 300 bp, Escherichia coli
attcatctgttgatcgtgggtgttggcctgatgagttatagcgatcccttgctgaaaataacatcatcattacgtcgcactgtggcggctatcgcactttaacgtttcgtgctgccccctcagtctatgcaatagaccataaactgcaaaaaaaagtccgctgataaggcttgaaaagttcatttccagacccatttttacatcgtagccgatgaggacgcgcctgatgggtgttctggctacctgacctgtccattgtggaaggtcttacattctcgctgatttcaggagctattgatt

SEQ ID NO: 22 - DNA, 300 bp, Escherichia coli
gtctggcattcgcccgtactcgtgataacgagatcgtggcaaaactgtttaacgaactgggcccgcgtttcgcgagccgtgccggtggttacactcgtattctgaagtgtggcttccgtgcaggcgacaacgcgccgatggcttacatcgagctggttgatcgttcagagaaagcagaagctgctgcagagtaatctgaagcaacgtaaaaaaacccgccccggcgggtttttttatacccgtagtatccccacttatctacaatagctgtactctttttgttcatcccctggagtattt

SEQ ID NO: 23 - DNA, 64 bp, Escherichia coli
caaaaaaaagtccgctgataaggcttgaaaagttcatttccagacccatttttacatcgtagcc

SEQ ID NO: 24 - DNA, 64 bp, Escherichia coli
attcaggcctattatgaaagctgcctgaacccgcaactgatcaccccttcagaaagccttatgc

SEQ ID NO: 25 - DNA, 65 bp, Escherichia coli
ccggtctgatgaccagcgatttatttcagaaaatcacgcggatgccgcaattgatgccgcaattg

SEQ ID NO: 26 - DNA, 63 bp, Escherichia coli
accaaaaatagttgcgcaaacatcttgaaattttgctaatgaccacaatataagctaaacgcg

SEQ ID NO: 27 - DNA, 64 bp, Escherichia coli
acaaaaaatttttgcatctcccccttgatgacgtggtttacgaccccatttagtagtcaaccgc

SEQ ID NO: 28 - DNA, 65 bp, Escherichia coli
gagagcaggatcacctgctctcgcttgaaattattctcccttgtccccatctctcccacatcctg

SEQ ID NO: 29 - DNA, 65 bp, Escherichia coli
cgtaacaacaaaagattgttatgcttgaaatatggtgatgccgtacccataacacagggactagc

SEQ ID NO: 30 - DNA, 64 bp, Escherichia coli
ttcacattaatctggtcaataaccttgaataattgagggatgacctcatttaatctccagtagc

SEQ ID NO: 31 - DNA, 64 bp, Escherichia coli
ctgcattcggctcgccgccgggggttgaaaccctcaaaatcccccccatctataattgcattat

SEQ ID NO: 32 - DNA, 65 bp, Escherichia coli
cagaattttttttctttttcccccttgaaggggcgaagcctcatccccatttctctggtcaccag

SEQ ID NO: 33 - DNA, 64 bp, Escherichia coli
ttaatttttcctctattctcggcgttgaatgtgggggaaacatccccatatactgacgtacatg

SEQ ID NO: 34 - DNA, 62 bp, Escherichia coli
tggtgacttacgcactatccagacttgaaaatagtcgcgtaacccatacgatgtgggtatcg

SEQ ID NO: 35 - DNA, 64 bp, Escherichia coli
ttgatgacaatgtgagtgcttcccttgaaaccctgaaactgatccccataataagcgaagttag

SEQ ID NO: 36 - DNA, 65 bp, Escherichia coli
tcgtattttttgcttacgttgggattgaaaacgggtcattctaccgccatctcccatatatcacc

SEQ ID NO: 37 - DNA, 55 bp, Escherichia coli
  misc_feature (1)..(2): spacer between conserved regions
nngaannnnatnnnntnntnncncttgaaannnngnnnnnnnnncnnccccatnt

SEQ ID NO: 38 - DNA, 65 bp, Escherichia coli
tgatgatgggagctggtttattgctttagttgtacgatgcaaaaaccaataaggaaacctgtgat

SEQ ID NO: 39 - DNA, 63 bp, Escherichia coli
ttttccgaatttagcccttaaatcatcaacaatgcgtgtggatgccattttcgcagacggcgc

SEQ ID NO: 40 - DNA, 64 bp, Escherichia coli
ctggtttggttaaaaattaaacacttgaaagtgtaatttccgtccccatatactaagcatcagt

SEQ ID NO: 41 - DNA, 64 bp, Escherichia coli
ataatctcaataattcaacttaatttgaaaattggaatatccatcacataacgacatgtcgcag

SEQ ID NO: 42 - DNA, 66 bp, Escherichia coli
tttgctgcgtcctgcattcaccagttgagtatcaagctttttgtccgccatgtcgggattcctgtt

SEQ ID NO: 43 - DNA, 65 bp, Escherichia coli
gcaacctgaaaaatgcctttcgtcttgaattgcccgtgcaaggtcgccatatggtgattgtggat

(19) United States
(12) Patent Application Publication
(10) Pub. No.: US 2003/0119094 A1
(43) Pub. Date: Jun. 26, 2003

(54) Solubility reporter gene constructs

(75) Inventors: Scott A. Lesley, San Diego, CA (US); Mark Knuth, El Cajon, CA (US)

Correspondence Address: TIMOTHY L. SMITH, GENOMICS INSTITUTE OF THE, NOVARTIS RESEARCH FOUNDATION, 10675 JOHN JAY HOPKINS DRIVE, SUITE E225, SAN DIEGO, CA, 92121-1127, US

(73) Assignee: IRM LLC

(21) Appl. No.: 09/990,099

(22) Filed: Nov. 21, 2001

Related U.S. Application Data
(60) Provisional application No. 60/324,833, filed on Sep. 24, 2001.

(51) Int. Cl. C12Q 1/68; C12P 21/02; C12N 5/06; C12N 15/85

(52) U.S. Cl. 435/69.1; 435/455; 435/320.1; 435/325

(57) ABSTRACT

The present invention provides polynucleotides that a protein solubility responsive promoter operatively liked to a reporter gene and a genetic reporter system comprising these polynucleotides together with an expression construct for a target protein. The invention also provides cells comprising polynucleotides of the invention and the genetic reporter system. These compositions are useful to monitor the solubility of a target protein in a cell and to identify mutations to the cell or mutations to a polynucleotide encoding the target protein that alters the solubility of the target protein. The invention further provides method to identify variations in a protein biosynthetic process that alter the solubility of a target protein and methods to screen an expression library of recombinant clones to identify clones that express soluble proteins. Finally, the invention discloses a novel method of identifying an antibiotic agent.

### CROSS-REFERENCE TO RELATED APPLICATIONS

This application claims priority to U.S. Provisional Application No. 60/324,833, filed Sep. 24, 2001. This application also claims priority to U.S. application Ser. No. 09/721,340, filed Nov. 21, 2000, which application was converted to US Provisional Application No. ______. Each of these applications is incorporated herein by reference for all purposes.

### BACKGROUND OF THE INVENTION

1. Field of the Invention

This invention pertains to the field of drug discovery and in particular, compositions and methods that aid the drug discovery process.

2. Background

While genetic engineering technology has provided the capability to modulate the expression of virtually any protein-encoding polynucleotide in a selected cell, it has been observed that purposeful manipulation of protein production in genetically modified cells often leads to the formation of incorrectly folded, biologically inactive protein molecules. In many cases, these mis-folded protein products form insoluble protein aggregates within the cytoplasm of the cell. Whether the purpose of the manipulation of expression of a target protein is to alter the phenotype of the cell, to provide a source of biologically active protein, or a source of protein that is suitable for structural analysis, these insoluble aggregates are biologically inactive, difficult to purify and difficult refold into an active configuration.

The biosynthesis of functional protein molecules occurs through translation of polypeptide-encoding messenger RNA molecules. The nascent polypeptide chain becomes folded into a three dimensional molecule. The ability of a protein to fold into a biologically active configuration is determined by the specific amino acid sequence of the protein and the conditions within the cell while the protein is being produced. In addition, accessory proteins called chaperones have been found to participate in the process of protein biosynthesis and can assist in the formation of properly folded protein molecules. Maxwell et al. (1999) Protein Science 8:1908-1911 have described a fusion protein construct that was useful to improve the solubility of several insoluble protein targets. However, this approach was limited in its utility by its dependence on fusion of a polynucleotide encoding the chloramphenicol acetyl transferase protein to a gene of interest. Thus there is a need for technology to monitor and control the folding of target proteins within a genetically modified cell.

Recent advances in the understanding of heat shock response proteins (Hsp) indicate that there exists a relationship between some of these proteins and protein folding. It is well known that cells which are subjected to elevated temperature respond by inducing the expression of a set of genes known as heat shock genes. The proteins encoded by these genes, the heat shock proteins, provide functions that help to control the deleterious effects of the elevated temperature and include chaperones and protease molecules. The heat shock response has been studied in detail in both eukaryotic and prokaryotic systems and is highly conserved throughout evolution. A thorough analysis of the genes induced by heat shock has been performed on the genome of the Gram negative bacterium E. coli to identify a set of genes induced by this stimulus (Richmond et al. (1999) Nucleic Acids Res. 27(19):3821-3835). The molecular basis of this response has also been studied in detail in E. coli (Liberek and Georgopoulos (1993) Proc. Nat'l. Acad. Sci. USA 90:11019-11023; and McCarty et al. (1996) J. Mol. Biol. 256:829-837). It has now been shown that alternative stressful stimuli such as altered pH, low oxygen (Lindquist (1986) Ann. Rev. Biochem. 55:1151-1191) or insoluble protein (Parcell and Sauer (1989) Genes and Development 3:1226-1232) also cause the induction of heat shock proteins. Studies on alternative conditions that induce the heat shock genes indicate that a common set of genes is induced by various stressful stimuli. Thus it appears that cells respond to a wide range of stressful conditions by producing a common set of proteins that include chaperones and protease molecules and that these proteins function to alleviate the deleterious effects of the harmful condition (see, e.g., Parsell et al. (1994) Nature 372(6505):475-478).

Several efforts have been made to take advantage of the heat shock response genes to monitor or protect against toxic conditions. For example, Farr (U.S. Pat. No. 5,589,337) describes a method for utilizing a stress response promoter fused to a gene that encodes an assayable product to characterize and quantify the toxicity of a compound. Also, Lindquist (U.S. Pat. No. 5,827,685) describes the use of the yeast Hsp 104 promoter and gene to protect cells against potentially toxic stress factors such as heat, alcohol and heavy metals. While these methods are useful for monitoring certain toxic stimuli they do not provide a convenient means to measure or improve the solubility of a range of protein products in cells. Therefore, a need exists for methods and reagents for determining and improving the solubility of proteins in cells. The present invention fulfills these and other needs.

### SUMMARY OF THE INVENTION

The present invention provides cells, reagents, and methods for determining whether a host cell expresses a polypeptide of interest in soluble or insoluble form. In some embodiments, the invention provides host cells that contain: a) a solubility reporter nucleic acid that includes a protein solubility responsive promoter operably linked to a reporter gene; and b) a target polypeptide-expressing nucleic acid that includes a polynucleotide that encodes a target polypeptide. Expression of the target polypeptide in an insoluble form causes a change in expression of the reporter gene. The solubility responsive promoter is upregulated when the target polypeptide is expressed in insoluble form in some embodiments of the invention; in other embodiments the solubility responsive promoter is downregulated when the target polypeptide is expressed in insoluble form. Arrays of two or more populations of such host cells are also provided; the host cells of each population differ in the target polypeptides expressed by the host cells.

The invention also provides methods for determining the solubility of a target polypeptide. These methods involve culturing host cells that contain: a) a solubility reporter nucleic acid that includes a protein solubility responsive promoter operably linked to a reporter gene; and b) a target polypeptide-expressing nucleic acid that includes a polynucleotide that encodes a target polypeptide under conditions in which the target polypeptide is expressed. The solubility of the expressed target polypeptide is then determined by detecting whether expression of the reporter gene is increased or decreased.

Additional embodiments of the invention provide methods for identifying mutations in a cell that alter the solubility of a target polypeptide. These methods involve: a) treating a cell with a mutagen; b) introducing into the cell a solubility reporter nucleic acid that includes a protein solubility responsive promoter operably linked to a reporter gene and a target polypeptide-expressing nucleic acid that includes a polynucleotide that encodes a target polypeptide; c) culturing the cell under conditions favorable for expression of the target polypeptide; d) measuring expression of the reporter gene; and e) comparing the level of expression of the reporter gene in the cell with the level observed in an unmutated cell that also contains the solubility reporter nucleic acid and the target polypeptide-expressing nucleic acid to identify a cell that comprises a mutation that alters the solubility of the target polypeptide.

In other embodiments, the invention provides methods for identifying alterations to a polynucleotide that encodes a target polypeptide that alter the solubility of the target polypeptide. These methods involve: a) altering a polynucleotide that encodes the target polypeptide to form an altered polynucleotide; b) introducing into a cell a solubility reporter nucleic acid that includes a protein solubility responsive promoter operably linked to a reporter gene, and a target polypeptide-expressing nucleic acid that includes the altered polynucleotide; c) culturing the cell under conditions favorable for expression of the target polypeptide; d) measuring the expression of the reporter gene; and e) comparing the level of expression of the reporter gene with the level observed in a cell with an unaltered polynucleotide that encodes the target polypeptide, to identify an alteration to the polynucleotide that changes the solubility of the encoded target polypeptide.

The invention also provides methods for identifying variations in a process for biosynthesis of a target polypeptide that alter the solubility of the target polypeptide. These methods involve culturing a host cell under alternative conditions in which the target polypeptide is expressed. The host cell includes: a) a solubility reporter nucleic acid that comprises a protein solubility responsive promoter operably linked to a reporter gene; and b) a target polypeptide-expressing nucleic acid that includes a polynucleotide that encodes a target polypeptide. Expression of the reporter gene by host cells grown under each of the alternative conditions is then compared to determine which condition results in a desired level of solubility of the target polypeptide.

Also provided by the invention are methods for screening an expression library to identify library members that express soluble target polypeptide. These methods involve: a) introducing a plurality of expression vectors that each include a polynucleotide that encodes a target polypeptide into a plurality of host cells to create an expression library, wherein the host cells contain a solubility reporter nucleic acid that includes a protein solubility responsive promoter operably linked to a reporter gene; b) culturing the host cells under conditions in which the target polypeptides are expressed; and c) detecting expression of the reporter gene, thereby identifying library members that express soluble target polypeptides.

The invention also provides methods for identifying an antibiotic agent. The methods involve: a) contacting a cell that contains a solubility reporter nucleic acid with a candidate antibiotic agent, wherein the solubility reporter nucleic acid includes a protein solubility responsive promoter operably linked to a reporter gene; and detecting the level of expression of the reporter gene. A change in the expression level of the reporter gene in a cell contacted with the candidate antibiotic agent, compared to reporter gene expression level in a cell which is not contacted with the candidate antibiotic agent, is indicative of an agent that inhibits protein folding in the cell.

The present invention also provides polynucleotides that include a protein solubility responsive promoter which is operably linked to a polynucleotide that encodes a detectable or selectable product. The polynucleotide can further comprise an expression construct for a target protein. This invention also provides a solubility reporter system that includes these solubility reporter polynucleotides together with an expression construct for a target protein. The invention also provides gene delivery vehicles and expression vectors and host or genetically modified cells containing at least polynucleotides of the invention and the genetic reporter system.

### BRIEF DESCRIPTION OF THE DRAWINGS

FIG. 1 shows the promoters of known heat shock genes that were induced during the expression of insoluble protein. The nucleotide sequences were aligned manually, allowing one gap in the sequence. Sequences are listed in decreasing level of induction of the most highly induced member of that operon. Promoters of the non-heat shock genes that were induced by translational misfolding are shown in the lower portion of the figure. Nucleotides that are conserved in RpoH recognition sequences are shown in gray shading.

FIGS. 2A-C shows a summary of screening results for 18 Thermatoga maritima proteins with pre-determined expression characteristics. The average relative β-galactosidase activity (FIG. 2A), Ni-HRP activity (FIG. 2B), and the resulting solubility scores (FIG. 2C) for the 18 T. maritima proteins are shown. Expression characteristics for the 18 proteins were previously determined by SDS-PAGE of both soluble and insoluble fractions.

FIG. 3 shows the relative β-galactosidase activity versus the relative Ni-HRP activity observed after expression of 186 T. maritima proteins in a reporter strain. Classification of each protein as soluble, insoluble, or mixed is based on SDS-PAGE performed on the soluble and insoluble lysates after the screen.

FIG. 4 shows an alignment of the secondary structure predictions and both predicted and identified domains of Rep68. Shown are Chou-Fasman secondary structure predictions of α-helical and β-sheet structures aligned with a Kyte-Doolittle plot of hydrophobicity based on the primary sequence of Rep68. Also aligned below are blocks representing the relative size and position of: the full-length Rep68 protein, the three predicted domains of Rep68, and the Rep68 domain identified by screening of randomly generated fragments of the rep68 gene. Solubility scores for the proteins are indicated.

### DETAILED DESCRIPTION

Definitions

Throughout this disclosure, various publications, patents and published patent specifications are referenced by an identifying citation. The disclosures of these publications, patents and published patent specifications are hereby incorporated by reference into the present disclosure to more fully describe the state of the art to which this invention pertains.

The practice of the present invention employs, unless otherwise indicated, conventional techniques of molecular biology (including recombinant techniques), microbiology, cell biology, biochemistry and immunology, which are within the skill of the art. Such techniques are explained fully in the literature. These methods are described in the following publications. See, e.g., Sambrook et al., MOLECULAR CLONING: A LABORATORY MANUAL, Third edition (2001); CURRENT PROTOCOLS IN MOLECULAR BIOLOGY (F. M. Ausubel et al. eds. (1987)); the series METHODS IN ENZYMOLOGY (Academic Press, Inc.); PCR: A PRACTICAL APPROACH (M. MacPherson et al., IRL Press at Oxford University Press (1991)); PCR 2: A PRACTICAL APPROACH (M. J. MacPherson, B. D. Haines and G. R. Taylor eds. (1995)); ANTIBODIES, A LABORATORY MANUAL (Harlow and Lane eds. (1988)); and ANIMAL CELL CULTURE (R. I. Freshney ed. (1987)).

As used herein, certain terms may have the following defined meanings.

As used in the specification and claims, the singular form “an” and “the” include plural references unless the context clearly dictates otherwise. For example, the term “a cell” includes a plurality of cells, including mixtures thereof.

The terms “polynucleotide” and “nucleic acid molecule” are used interchangeably to refer to polymeric forms of nucleotides of any length. The polynucleotides may contain deoxyribonucleotides, ribonucleotides, and/or their analogs. Polynucleotides may have any three-dimensional structure, and may perform any function, known or unknown. The term “polynucleotide” includes, for example, single-double-stranded and triple helical molecules, a gene or gene fragment, exons, introns, mRNA, tRNA, rRNA, ribozymes, cDNA, recombinant polynucleotides, branched polynucleotides, plasmids, vectors, isolated DNA of any sequence, isolated RNA of any sequence, nucleic acid probes, and primers. A nucleic acid molecule may also comprise modified nucleic acid molecules.

The term “peptide” is used in its broadest sense to refer to a compound of two or more subunit amino acids, amino acid analogs, or peptidomimetics. The subunits may be linked by peptide bonds. In another embodiment, the subunit may be linked by other bonds, e.g. ester, ether, etc. As used herein the term “amino acid” refers to either natural and/or unnatural or synthetic amino acids, including glycine and both the D or L optical isomers, and amino acid analogs and peptidomimetics. A peptide of three or more amino acids is commonly called an oligopeptide if the peptide chain is short. If the peptide chain is long (e.g., longer than about 10-20 amino acids), the peptide is commonly called a polypeptide or a protein.

The term “genetically modified” means containing and/or expressing a foreign gene or nucleic acid sequence which in turn, modifies the genotype or phenotype of the cell or its progeny. In other words, it refers to any addition, deletion or disruption to a cell's endogenous polynucleotides. The term “heterologous” also refers to a polynucleotide or polypeptide that is not naturally associated with a particular cell or cellular components. For example, a promoter that is heterologous to a particular host cell is not found in a naturally occurring cell of that species. Similarly, a promoter that is heterologous to a particular protein-encoding polynucleotide is not found attached to that particular polynucleotide in a naturally occurring cell. The term “recombinant” is sometimes used to refer to nucleic acids that include polynucleotides that are not associated with each other in cells that are unmodified by recombinant methods.

As used herein, “expression” refers to the process by which polynucleotides are transcribed into mRNA and translated into peptides, polypeptides, or proteins. If the polynucleotide is derived from genomic DNA, expression may include splicing of the mRNA, if an appropriate eukaryotic host is selected. Regulatory elements required for expression include promoter sequences to bind RNA polymerase and translation initiation sequences for ribosome binding. For example, a bacterial expression vector includes a promoter such as the lac promoter and for transcription and translation initiation the Shine-Dalgarno sequence and the start codon ATG (Sambrook et al. (2001) supra). Similarly, a eukaryotic expression vector includes a heterologous or homologous promoter for RNA polymerase II, a downstream polyadenylation signal, the start codon AUG, and a termination codon for detachment of the ribosome. Such vectors can be obtained commercially or assembled by the sequences described in methods well known in the art, for example, the methods described below for constructing vectors in general.

A “promoter” is a region on a DNA molecule to which an RNA polymerase binds and initiates transcription. The nucleotide sequence of the promoter determines both the nature of the enzyme that attaches to it and the rate of RINA synthesis. In the present disclosure the term “promoter” is used to mean a polynucleotide that includes not only the RNA polymerase binding site but also all other contiguous sequence elements that interact with factors which modulate transcription initiation, such as repressors or inducers of transcription. Thus a “promoter” as defined here, is a polynucleotide that contains all of the sequence information required to regulate gene expression in the same way as the native element in the chromosome.

The term “protein solubility responsive promoter” means a promoter element that is either induced or repressed in a cell in response to an increased concentration of insoluble protein in the cytoplasm.

“Under transcriptional control” is a term well understood in the art and indicates that transcription of a polynucleotide sequence, usually a DNA sequence, depends on its being operatively linked to an element which contributes to the initiation of, or promotes, transcription. “Operatively linked” refers to a juxtaposition wherein the elements are in an arrangement allowing them to function.

The term “expression construct” means a polynucleotide comprising a promoter element operatively linked to a gene. The expression construct can be formatted in a variety of ways such as in a gene delivery vehicle or inserted into a chromosome of a cell. The term is intended to refer to promoter-gene fusions produced by any method including, but not limited to recombinant DNA techniques, homologous recombination, targeted insertion of a gene or promoter element or random insertion of a gene or promoter element.

A “gene delivery vehicle” is defined as any molecule that can carry inserted polynucleotides into a host cell. Examples of gene delivery vehicles are liposomes, biocompatible polymers, including natural polymers and synthetic polymers; lipoproteins; polypeptides; polysaccharides; lipopolysaccharides; artificial viral envelopes; metal particles; and bacteria, viruses, such as baculovirus, adenovirus and retrovirus, bacteriophage, cosmid, plasmid, fungal vectors and other recombination vehicles typically used in the art which have been described for expression in a variety of eukaryotic and prokaryotic hosts, and may be used for gene therapy as well as for simple protein expression.

“Gene delivery,” “gene transfer,” and the like as used herein, are terms referring to the introduction of an exogenous polynucleotide (sometimes referred to as a “transgene”) into a host cell, irrespective of the method used for the introduction. Such methods include a variety of well-known techniques such as vector-mediated gene transfer (by, e.g., viral infection/transfection, or various other protein-based or lipid-based gene delivery complexes) as well as techniques facilitating the delivery of “naked” polynucleotides (such as electroporation, “gene gun” delivery and various other techniques used for the introduction of polynucleotides). The introduced polynucleotide may be stably or transiently maintained in the host cell. Stable maintenance typically requires that the introduced polynucleotide either contains an origin of replication compatible with the host cell or integrates into a replicon of the host cell such as an extrachromosomal replicon (e.g., a plasmid) or a nuclear or mitochondrial chromosome. A number of vectors are known to be capable of mediating transfer of genes to mammalian cells, as is known in the art and described herein.

A “viral vector” is defined as a recombinantly produced virus or viral particle that comprises a polynucleotide to be delivered into a host cell, either in vivo, ex vivo or in vitro. Examples of viral vectors include retroviral vectors, adenovirus vectors, adeno-associated virus vectors and the like. In aspects where gene transfer is mediated by a retroviral vector, a vector construct refers to the polynucleotide comprising the retroviral genome or part thereof, and a therapeutic gene. As used herein, “retroviral mediated gene transfer” or “retroviral transduction” carries the same meaning and refers to the process by which a gene or nucleic acid sequences are stably transferred into the host cell by virtue of the virus entering the cell and integrating its genome into the host cell genome. The virus can enter the host cell via its normal mechanism of infection or be modified such that it binds to a different host cell surface receptor or ligand to enter the cell. As used herein, retroviral vector refers to a viral particle capable of introducing exogenous nucleic acid into a cell through a viral or viral-like entry mechanism.

Retroviruses carry their genetic information in the form of RNA; however, once the virus infects a cell, the RNA is reverse-transcribed into the DNA form which integrates into the genomic DNA of the infected cell. The integrated DNA form is called a provirus.

In aspects where gene transfer is mediated by a DNA viral vector, such as an adenovirus (Ad) or adeno-associated virus (AAV), a vector construct refers to the polynucleotide comprising the viral genome or part thereof, and a transgene. Adenoviruses (Ads) are a relatively well characterized, homogenous group of viruses, including over 50 serotypes. See, e.g., WO 95/27071. Ads are easy to grow and do not require integration into the host cell genome. Recombinant Ad-derived vectors, particularly those that reduce the potential for recombination and generation of wild-type virus, have also been constructed. See, WO 95/00655 and WO 95/11984. Wild-type AAV has high infectivity and specificity integrating into the host cell's genome. See, Hermonat and Muzyczka (1984) Proc. Nat'l. Acad. Sci. USA 81:6466-6470 and Lebkowski et al. (1988) Mol. Cell. Biol. 8:3988-3996.

Vectors that contain both a promoter and a cloning site into which a polynucleotide can be operatively linked are well known in the art. Such vectors are capable of transcribing RNA in vitro or in vivo, and are commercially available from sources such as Stratagene (La Jolla, Calif.) and Promega Biotech (Madison, Wis.). In order to optimize expression and/or in vitro transcription, it may be necessary to remove, add or alter 5′ and/or 3′ untranslated portions of the clones to eliminate extra, potential inappropriate alternative translation initiation codons or other sequences that may interfere with or reduce expression, either at the level of transcription or translation. Alternatively, consensus ribosome binding sites can be inserted immediately 5′ of the start codon to enhance expression.

Gene delivery vehicles also include several non-viral vectors, including DNA/liposome complexes, and targeted viral protein-DNA complexes. Liposomes that also comprise a targeting antibody or fragment thereof can be used in the methods of this invention. To enhance delivery to a cell, the nucleic acid or proteins of this invention can be conjugated to antibodies or binding fragments thereof which bind cell surface antigens, e.g., TCR, CD3 or CD4.

As used herein, a “reporter gene” is a polynucleotide encoding a protein whose expression by a cell can be detected and quantified. Thus, a measurement of the level of expression of the reporter is indicative of the level of activation of the promoter element that directs expression of the reporter gene. Such detection includes, for example, selection for the presence of reporter gene expression by placing cells that contain the reporter gene under selective conditions.

“Hybridization” refers to a reaction in which one or more polynucleotides react to form a complex that is stabilized via hydrogen bonding between the bases of the nucleotide residues. The hydrogen bonding may occur by Watson-Crick base pairing, Hoogstein binding, or in any other sequence-specific manner. The complex may comprise two strands forming a duplex structure, three or more strands forming a multi-stranded complex, a single self-hybridizing strand, or any combination of these. A hybridization reaction may constitute a step in a more extensive process, such as the initiation of a PCR reaction, or the enzymatic cleavage of a polynucleotide by a ribozyme.

Examples of stringent hybridization conditions include: incubation temperatures of about 25° C. to about 37° C.; hybridization buffer concentrations of about 6×SSC to about 10×SSC; formamide concentrations of about 0% to about 25%; and wash solutions of about 6×SSC. Examples of moderate hybridization conditions include: incubation temperatures of about 40° C. to about 50° C.; buffer concentrations of about 9×SSC to about 2×SSC; formamide concentrations of about 30% to about 50%; and wash solutions of about 5×SSC to about 2×SSC. Examples of high stringency conditions include: incubation temperatures of about 55° C. to about 68° C.; buffer concentrations of about 1×SSC to about 0.1×SSC; formamide concentrations of about 55% to about 75%; and wash solutions of about 1×SSC, 0.1×SSC, or deionized water. In general, hybridization incubation times are from 5 minutes to 24 hours, with 1, 2, or more washing steps, and wash incubation times are about 1, 2, or 15 minutes. SSC is 0.15 M NaCl and 15 mM citrate buffer. It is understood that equivalents of SSC using other buffer systems can be employed.

A polynucleotide or polynucleotide region (or a polypeptide or polypeptide region) has a certain percentage (for example, 80%, 85%, 90%, or 95%) of “sequence identity” to another sequence means that, when aligned, that percentage of bases (or amino acids) are the same in comparing the two sequences. This alignment and the percent homology or sequence identity can be determined using software programs known in the art, for example those described in CURRENT PROTOCOLS IN MOLECULAR BIOLOGY (F. M. Ausubel et al., eds., 1987) Supplement 30, section 7.7.18, Table 7.7.1. A preferred program for aligning polynucleotide and polypeptide sequences to determine percent homology is CLUSTALW, using default parameters. This program is available on the world wide web at a variety of sites such as the Institute for Biological Computing at Washington University in Saint Louis, Mo. (www.ibc.wustl.edulmsalclustal.html), the Human Genome Sequencing Center of the Baylor College of Medicine in Houston, Tex. (dot.imgen.bcm.tmc.edu:9331/multi-align/multi-align.html) and the Pasteur Institute in Paris, France (bioweb.pasteur.fr/seqanal/interfaces/clustalw-simple.html)

A “biological equivalent” of a reference polynucleotide is one characterized by possessing at least 75%, or at least 80%, or at least 90% or at least 95% sequence identity as determined using a sequence alignment program under default parameters, correcting for ambiguities in the sequence data and changes in nucleotide sequence that do not alter function. A “biologically equivalent” polynucleotide can also be isolated by hybridization under moderate or stringent hybridization conditions. In addition to sequence similarity or hybridization with reference polynucleotides, the biologically equivalent polynucleotide has the same or similar biological function as the reference polynucleotide.

A variety of software programs are available in the art to identify biologically equivalent polynucleotides without an undue amount of experimentation. Non-limiting examples of these programs are BLAST family programs including BLASTN, BLASTP, BLASTX, TBLASTN, and TBLASTX (BLAST is available from the worldwide web at http://www.ncbi.nlm.nih.gov/BLASTI), FastA, Compare, DotPlot, BestFit, GAP, FrameAlign, ClustalW, and PileUp. These programs can be obtained commercially in a comprehensive package of sequence analysis software such as GCG Inc.'s Wisconsin Package. Other similar analysis and alignment programs can be purchased from various providers such as DNA Star's MegAlign, or the alignment programs in GeneJockey. Alternatively, sequence analysis and alignment programs can be accessed through the world wide web at sites such as the CMS Molecular Biology Resource at www.sdsc.edu/ResTools/cmshp.html. Any sequence database that contains DNA or protein sequences corresponding to a gene or a segment thereof can be used for sequence analysis. Commonly employed databases include but are not limited to GenBank, EMBL, DDBJ, PDB, SWISS-PROT, EST, STS, GSS, and HTGS. Sequence similarity can be discerned by aligning the tag sequence against a DNA sequence database. Alternatively, the tag sequence can be translated into six reading frames; the predicted peptide sequences of all possible reading frames are then compared to individual sequences stored in a protein database such as s done using the BLASTX program.

Parameters for determining the extent of homology set forth by one or more of the aforementioned alignment programs are well established in the art. They include but are not limited to p value, percent sequence identity and the percent sequence similarity. P value is the probability that the alignment is produced by chance. For a single alignment, the p value can be calculated according to Karlin et al. (1990) Proc. Nat'l. Acad. Sci. USA 87: 2246. For multiple alignments, the p value can be calculated using a heuristic approach such as the one programmed in BLAST. Percent sequence identify is defined by the ratio of the number of nucleotide or amino acid matches between the query sequence and the known sequence when the two are optimally aligned. The percent sequence similarity is calculated in the same way as percent identity except one scores amino acids that are different but similar as positive when calculating the percent similarity.

“In vivo” gene delivery, gene transfer, gene therapy and the like as used herein, are terms referring to the introduction of a vector comprising an exogenous polynucleotide directly into the body of an organism, such as a human or non-human mammal, whereby the exogenous polynucleotide is introduced to a cell of such organism in vivo.

The term “isolated” means separated from constituents, cellular and otherwise, in which the polynucleotide, peptide, polypeptide, protein, antibody, or fragments thereof, are normally associated with in nature. For example, with respect to a polynucleotide, an isolated polynucleotide is one that is separated from the 5′ and 3′ sequences with which it is normally associated in the chromosome. As is apparent to those of skill in the art, a non-naturally occurring polynucleotide, peptide, polypeptide, protein, antibody, or fragments thereof, does not require “isolation” to distinguish it from its naturally occurring counterpart. In addition, a “concentrated”, “separated” or “diluted” polynucleotide, peptide, polypeptide, protein, antibody, or fragments thereof, is distinguishable from its naturally occurring counterpart in that the concentration or number of molecules per volume is greater than “concentrated” or less than “separated” than that of its naturally occurring counterpart. A polynucleotide, peptide, polypeptide, protein, antibody, or fragments thereof, which differs from the naturally occurring counterpart in its primary sequence or for example, by its glycosylation pattern, need not be present in its isolated form since it is distinguishable from its naturally occurring counterpart by its primary sequence, or alternatively, by another characteristic such as glycosylation pattern. Although not explicitly stated for each of the inventions disclosed herein, it is to be understood that all of the above embodiments for each of the compositions disclosed below and under the appropriate conditions, are provided by this invention. Thus, a non-naturally occurring polynucleotide is provided as a separate embodiment from the isolated naturally occurring polynucleotide. A protein produced in a bacterial cell is provided as a separate embodiment from the naturally occurring protein isolated from a eucaryotic cell in which it is produced in nature.

“Host cell,” or “genetically modified cell” are intended to include any individual cell or cell culture which can be or have been recipients for vectors or the incorporation of exogenous nucleic acid molecules, polynucleotides and/or proteins. It also is intended to include progeny of a single cell, and the progeny may not necessarily be completely identical (in morphology or in genomic or total DNA complement) to the original parent cell due to natural, accidental, or deliberate mutation. The cells may be procaryotic or eucaryotic, and include but are not limited to bacterial cells, yeast cells, animal cells, and mammalian cells, e.g., murine, rat, simian or human.

A “subject” is a vertebrate, preferably a mammal, more preferably a human. Mammals include, but are not limited to, murines, simians, humans, farm animals, sport animals, and pets.

A “control” is an alternative subject or sample used in an experiment for comparison purpose. A control can be “positive” or “negative.” For example, where the purpose of the experiment is to determine a correlation of an altered expression level of a gene with a particular type of cancer, it is generally preferable to use a positive control (a subject or a sample from a subject, carrying such alteration and exhibiting syndromes characteristic of that disease), and a negative control (a subject or a sample from a subject lacking the altered expression and clinical syndrome of that disease).

The term “culturing” refers to the in vitro propagation of cells or organisms on or in media of various kinds. It is understood that the descendants of a cell grown in culture may not be completely identical (morphologically, genetically, or phenotypically) to the parent cell. By “expanded” is meant any proliferation or division of cells. A “composition” is intended to mean a combination of active agent and another compound or composition, inert (for example, a detectable agent or label) or active, such as an adjuvant.

A “pharmaceutical composition” is intended to include the combination of an active agent with a carrier, inert or active, making the composition suitable for diagnostic or therapeutic use in vitro, in vivo or ex vivo.

As used herein, the term “pharmaceutically acceptable carrier” encompasses any of the standard pharmaceutical carriers, such as a phosphate buffered saline solution, water, and emulsions, such as an oil/water or water/oil emulsion, and various types of wetting agents. The compositions also can include stabilizers and preservatives. For examples of carriers, stabilizers and adjuvants, see Martin REMINGTON'S PHARM. SCI., 15th Ed. (Mack Publ. Co., Easton (1975)).

An “effective amount” is an amount sufficient to effect beneficial or desired results. An effective amount can be administered in one or more administrations, applications or dosages.

“Solid growth media” is growth media appropriate to the organism being cultured which contains agar at sufficient concentration to provide a solid surface for the purpose of plating cultures for clonal populations of cells.

“Indicator dyes” refer to chemicals which react with the product of the reporter gene to produce a compound with altered properties that can easily be assayed. An example of a suitable indicator dye is X-gal which reacts with beta-galactosidase, the gene product of the lacZ reporter, to produce a blue precipitate.

### DESCRIPTION OF THE PREFERRED EMBODIMENTS

The invention provides solubility reporter gene constructs that allow one to readily distinguish whether a protein is produced by a cell in an insoluble form or a soluble form. Also provided are reporter host cells for use in identifying proteins or protein domains that are produced in soluble form, as well as methods for determining the protein solubility state in a cell. In further embodiments, the invention provides high-throughput methods for determining the solubility state of a target protein that is expressed in a cell.

Solubility Reporter Gene Constructs

This invention provides host cells that contain solubility reporter constructs that include a promoter that is induced or repressed depending upon whether insoluble proteins are present in a cell that contains the promoter. These protein solubility responsive promoters are preferably linked to a polynucleotide that encodes a gene product that is readily detectable when expressed in a cell. When a solubility reporter gene construct that includes a promoter that is upregulated by insoluble proteins is present in a cell, for example, the presence of insoluble protein will result in an increase in the level of the reporter gene product.

To identify suitable promoters for use in a particular species, one can compare gene expression profiles from cells of that species that express a protein that is known to be expressed in an insoluble form to cells that do not express an insoluble protein. For example, the control cells can express a protein that is found in soluble form. Once one or more genes that are differentially expressed depending upon whether an insoluble or soluble protein is expressed, a region upstream of that gene can be cloned and used to construct a solubility reporter construct. The length of the polynucleotide that includes upstream region will sometimes vary depending upon the particular gene and/or species. Once an upstream region is cloned, one can readily test its functionality by operably linking the upstream region to a reporter structural gene, introducing the construct into a host cell, and expressing a protein that is known to be expressed in insoluble form. Promoter sequences responsive to misfolded protein can be identified by, for example, Affymetrix GeneChip®, cDNA array, reporter screening, and other approaches that are known to those of skill in the art.

The protein solubility responsive promoter can be a prokaryotic or a eukaryotic promoter. A promoter that is functional in the particular host cell of interest is utilized. For example, for use in bacterial host cells, one can isolate the protein solubility responsive promoter from a Gram negative or a Gram positive bacterium. Such Gram negative bacteria include, for example, members of the family Enterobacteriaceae. Examples of the members of the Enterobacteriaceae are the genera Escherichia, Salmonella, Shigella, Klebsiella or Enterobacter. Suitable prokaryotic cells include, but are not limited to Salmonella typhomurium, Bacillus subtilis and Streptomyces lividans. One suitable species is a promoter element isolated from the Gram negative bacterium E. coli. Specific examples of suitable E. coli promoters include, for example, promoters from the following genes: kgtP gene (b2587; SEQ ID NO:1), gene b3913 (SEQ ID NO:2), proP (b4111; SEQ ID NO:3), exbB (b3006; SEQ ID NO:4), yegG (b2812; SEQ ID NO:5), yojH (b2210; SEQ ID NO:6), ybeD (b0631); SEQ ID NO:7, yciS (b1279; SEQ ID NO:8), yagU (b0287; SEQ ID NO:9), ftsJ (b3179; SEQ ID NO:10), grpE (b2614; SEQ ID NO:11), htpX (b1829; SEQ ID NO:12), clpB (b2592; SEQ ID NO:13), fxsA (b4140; SEQ ID NO:14), hslV (b3932; SEQ ID NO:15), clpP (b0437; SEQ ID NO:16), htpG (b0473; SEQ ID NO:17), dnaK (b0014; SEQ ID NO:18), yccV (b0966, SEQ ID NO:19), yrfG (b3399; SEQ ID NO:20), ibpA (b3687; SEQ ID NO:21), and yhdN (b3293; SEQ ID NO:22).

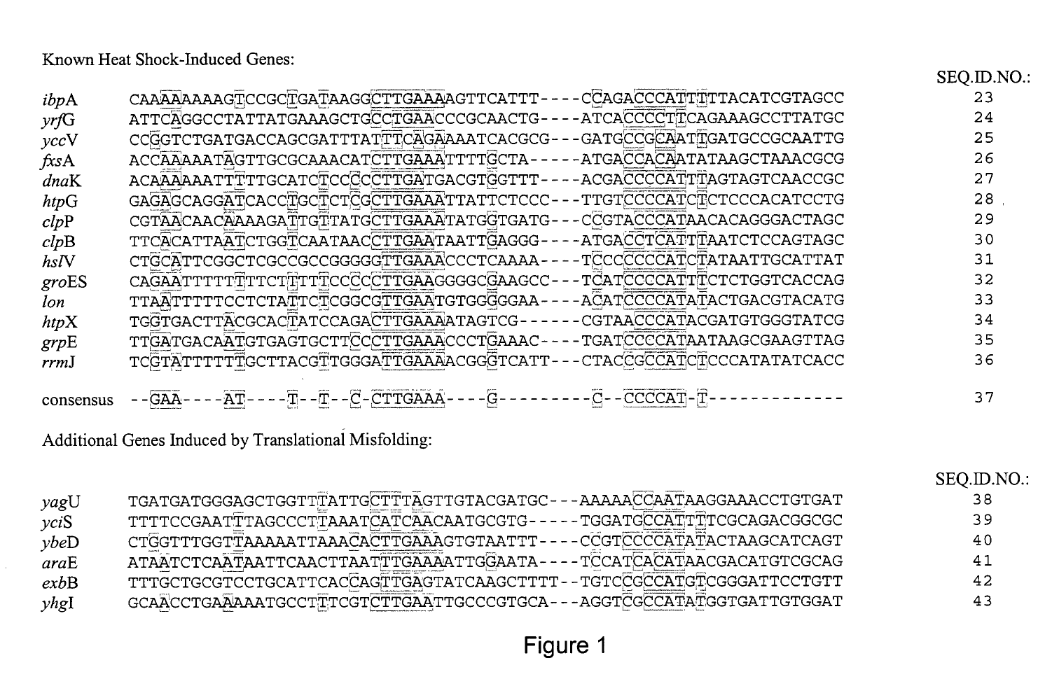

In some embodiments, the protein solubility responsive promoters include an RpoH recognition site. Examples of such promoters are shown in FIG. 1, and as SEQ ID NOS:23-43.

This invention also encompasses the use of biologically equivalent polynucleotides to the sequences provided in Seq. ID. Nos. 1-43, which can be identified using sequence homology searches or hybridization under moderate or stringent hybridization conditions as defined above. Several embodiments of biologically equivalent polynucleotides are within the scope of this invention, e.g., those characterized by possessing at least 75%, or at least 80%, or at least 90% or at least 95% sequence homology as determined using a sequence alignment program under default parameters correcting for ambiguities in the sequence data, and changes in nucleotide sequence that do not alter function. Biological equivalents also includes those that hybridize under conditions of moderate or stringent conditions to the sequences of Seq. ID. Nos. 1-43, or their respective complements. Such polynucleotides can be tested according to the methods of the invention to identify those that exhibit the desired protein solubility responsiveness.

For use in eukaryotic cells, a protein solubility responsive promoter is generally obtained from a eukaryotic gene. Many eukaryotic heat shock and other stress-induced genes are known to those of skill in the art.

The invention provides methods for testing promoters from these and other genes to determine whether the promoters are differentially regulated in response to the presence of an insoluble protein in the cell. These methods involve culturing a host cell that includes a solubility reporter nucleic acid that comprises a putative protein solubility responsive promoter operably linked to a reporter gene. The host cell also contains a target polypeptide-expressing nucleic acid that includes a polynucleotide that encodes a target polypeptide. The host cell is cultured under conditions in which the target polypeptide is expressed in insoluble form. The level of expression of the reporter gene is then detected to determine whether the putative protein solubility responsive promoter is differentially regulated in response to expression of an insoluble polypeptide in the host cell.

Suitable eukaryotic cells include, for example, mammalian, insect, or plant cells or microorganisms, such as, for example, yeast cells, or fungal cells. Examples of suitable cells include, for example, Azotobacter sp. (e.g., A. vinelandii), Pseudomonas sp., Rhizobium sp., Erwinia sp., Escherichia sp. (e.g., E. coli), and Klebsiella sp., among many others. Yeast cells can be of any of several genera, including Saccharomyces (e.g., S. cerevisiae), Candida (e.g., C. utilis, C. parapsilosis, C. krusei, C. versatilis, C. lipolytica, C. zeylanoides, C. guilliermondii, C. albicans, and C. humicola), Pichia (e.g., P. farinosa and P. ohmeri), Torulopsis (e.g., T. candida, T. sphaerica, T. xylinus, T. famata, and T. versatilis), Debaryomyces (e.g., D. subglobosus, D. cantarellii, D. globosus, D. hansenii, and D. japonicus), Zygosaccharomyces (e.g., Z. rouxii and Z. bailii), Kluyveromyces (e.g., K. marxianus), Hansenula (e.g., H. anomala and H. jadinii), and Brettanomyces (e.g., B. lambicus and B. anomalus). Additional non-limiting examples of suitable eukaryotic cells include Jurkat cells and NIH3T3 cells.

The protein solubility responsive promoters identified above are operatively linked to a reporter gene that functions to identify the presence or absence of soluble protein in the cell cytoplasm. The reporter genes include a polynucleotide that encodes a selectable or detectable polypeptide. Examples of genes useful as “reporter genes” include, but are not limited genes that encode a metabolic enzyme, an antibiotic resistance factor, a luminescent protein (e.g., luciferase), or a fluorescent protein. Such reporter genes are well known in the art and particular examples are described in Wood (1995) Curr. Opin. Biotechnol. 6(1):50-58. In one aspect, the metabolic enzyme is β-galactosidase. In other aspects, the metabolic gene is a gene that complements an auxotrophic mutation in a host cell and allows growth of cells that express the gene on selective media.

Methods for detecting and quantitating reporter expression are commonly based on measuring the activity of the protein encoded by the reporter. A wide variety of appropriate detectable markers are known in the art, including fluorescent, radioactive, enzymatic or other ligands, such as avidin/biotin, which are capable of giving a detectable signal. In preferred embodiments, one will likely desire to employ a fluorescent label or an enzyme tag, such as urease, alkaline phosphatase or peroxidase, instead of radioactive or other environmentally undesirable reagents. In the case of enzyme tags, colorimetric indicator substrates are known which can be employed to provide a means visible to the human eye or spectrophotometrically, to identify specific hybridization with complementary nucleic acid-containing samples.

When the reporter is an enzyme, a substrate for the enzyme which is metabolized to produce a measurable product can be used. For example, the β-galactosidase substrate X-gal, which is cleaved by this enzyme to produce a blue reaction product, is frequently used to assay β-galactosidase reporter expression. (Miller J. ed. (1992) A Short Course in Bacterial Genetics: A Laboratory Manual and Handbook for Escherichia Coli and Related Bacteria, Cold Spring Harbor Laboratory, Cold Spring Harbor, N.Y. Alternatively, the β-galactosidase substrate o-nitrophyl-B-D-galactopyranoside (ONPG), which is metabolized by β-galactosidase to produce a compound with a yellow color. The quantity of enzyme is determined by measuring optical density of the colored compound spectrophotometrically or with an ELISA reader. The absorbance is read at 420 nm (Miller J. H. ed. (1972) Experiments in Molecular Genetics, Cold Spring Harbor Laboratory, Cold Spring Harbor, N.Y.). Other commonly used reporter genes are the antibiotic resistance factor chloramphenicol acetyl transferase (CAT), the firefly luciferase gene, and the jellyfish green fluorescent protein (Valdivia and Falkow (1997) Trends Microbiol. 5(9):360-363; Naylor (1999) Biochem. Pharmacol. 58(5):749-757; Himes and Shannon (2000) Methods Mol. Biol. 130:165-174). In addition, a variety of alternative proteins can also be used as reporters based on their ability to be detected and quantitated. Assays to measure the expression levels of such genes are well developed and are commonly practiced by those of ordinary skill (Rosenthal (1987) Methods Enzymology 152:704-720; Davey et al. (1995) Methods Mol. Biol. 49:143-148; and Bronstein et al. (1994) Anal. Biochem. 219(2):169-181).

Polynucleotides that encode useful reporter genes are available from a variety of commercial suppliers of molecular biology reagents such as LifeTechnologies Inc. (Gaithersburg, Md.), Clontech Inc. (Palo Alto, Calif.), Promega Inc. (Madison, Wis.), Invitrogen Inc. (Carlsbad, Calif.), and Strategene Inc. (San Diego, Calif.). In addition, plasmid vectors comprising reporter gene sequences are available from the American Type Culture Collection and genetic repositories such as the E. coli strain collection at Yale University.

The solubility reporter nucleic acids of the invention can comprise additional sequences, such as coding sequences within the same transcription unit, controlling elements such as ribosome binding sites, and polyadenylation sites, additional transcription units under control of the same or a different promoter, sequences that permit cloning, expression, and transformation of a host cell, and any such construct as may be desirable to provide embodiments of this invention. In some embodiments, the solubility reporter nucleic acids include a polynucleotide that encodes a signal peptide that directs a detectable polypeptide encoded by the reporter gene to a surface of the host cell. The detectable polypeptide can then be detected by, e.g., a cell sorter. For example, if the reporter gene encodes a fluorescent protein, which is displayed on the surface of the cell upon expression, one can utilize a fluorescence activated cell sorter to separate cells that express the reporter gene from those that do not.

The solubility reporter nucleic acids can also include a polynucleotide that encodes a molecular tag which can facilitate separation of a host cell that expresses the reporter gene from a host cell that does not express the reporter gene. For example, an epitope for an antibody can function as a molecular tag; cells that express the reporter gene can then be immobilized by contacting the cells with a solid support to which is attached antibodies that specifically recognize the epitope. Other suitable molecular tags are well known to those of skill in the art, and include, for example, a poly-histidine tag, or a FLAG™ peptide. If the particular protein solubility responsive promoter in use is upregulated in response to expression of a target polypeptide in insoluble form, cells that express the insoluble target polypeptide will be immobilized on the support. Conversely, if the particular protein solubility responsive promoter in use is downregulated in response to expression of a target polypeptide in insoluble form, cells that express the target polypeptide in soluble form will be immobilized on the support.

The invention also provides a reporter system comprising: a) an isolated polynucleotide containing at least a protein solubility responsive promoter operatively linked to a reporter gene, and b) an expression construct that directs the expression of a target gene. The expression construct can be either on a separate polynucleotide from the promoter and reporter gene or the expression construct can be part of a single polynucleotide that also contains the protein solubility responsive promoter and reporter gene. Thus in a particular embodiment of the invention the reporter system comprises an isolated polynucleotide with a protein solubility responsive promoter operatively linked to a reporter gene, wherein the isolated polynucleotide further comprises an expression construct.

The present invention also provides gene delivery vehicles suitable for delivery and/or expression of a polynucleotide of the invention into cells (whether in vivo, ex vivo, or in vitro) containing the polynucleotides of this invention. A polynucleotide of the invention can be contained within a cloning or expression vector. These vectors (especially expression vectors) can in turn be manipulated to assume any of a number of forms which may, for example, facilitate delivery to and/or entry into a cell. Examples of suitable expression and delivery vehicles are provided above.

This invention also provides host or genetically modified cells containing the protein solubility reporter constructs described above, as well as a target polypeptide-expressing nucleic acid that includes a polynucleotide that encodes a target polypeptide identified above. Arrays of cells are also provided, in which the cells of each population differ in the target polypeptides expressed by the cells. For example, the polypeptides can differ due to amino acid substitutions, deletions, or insertions compared to a reference amino acid sequence. Alternatively, the target polypeptides expressed by the populations of host cells can be different fragments of a larger polypeptide.

The polynucleotides and sequences embodied in this invention can be obtained using chemical synthesis, recombinant cloning methods, PCR, or any combination thereof. The PCR technology is the subject matter of U.S. Pat. Nos. 4,683,195; 4,800,159; 4,754,065; and 4,683,202 and described in PCR: THE POLYMERASE CHAIN REACTION (Mullis et al. eds, Birkhauser Press, Boston (1994)) or MacPherson et al. (1991) and (1995), supra, and references cited therein. Alternatively, one of skill in the art can use the sequences provided herein and a commercial DNA synthesizer to replicate the DNA. Accordingly, this invention also provides a process for obtaining the polynucleotides of this invention by providing the linear sequence of the polynucleotide, nucleotides, appropriate primer molecules, chemicals such as enzymes and instructions for their replication and chemically replicating or linking the nucleotides in the proper orientation to obtain the polynucleotides. In a separate embodiment, these polynucleotides are further isolated. Still further, one of skill in the art can insert the polynucleotide into a suitable replication vector and insert the vector into a suitable host cell (prokaryotic or eukaryotic) for replication and amplification. The DNA so amplified can be isolated from the cell by methods well known to those of skill in the art. A process for obtaining polynucleotides by this method is further provided herein as well as the polynucleotides so obtained.

RNA can be obtained by first inserting a DNA polynucleotide into a suitable host cell. The DNA can be inserted by any appropriate method, e.g., by the use of an appropriate gene delivery vehicle (e.g., liposome, plasmid or vector) or by electroporation. When the cell replicates and the DNA is transcribed into RINA; the RNA can then be isolated using methods well known to those of skill in the art, for example, as set forth in Sambrook et al. (2001) supra. For instance, mRNA can be isolated using various lytic enzymes or chemical solutions according to the procedures set forth in Sambrook et al. (2001), supra or extracted by nucleic-acid-binding resins following the accompanying instructions provided by manufacturers.

Compositions containing a carrier and the polynucleotides and sequences of this invention, in isolated form or contained within a vector or host or genetically modified cell are further provided herein. When these compositions are to be used pharmaceutically, they are combined with a pharmaceutically acceptable carrier.

The polynucleotides, reporter systems and cells are useful in the methods described below.

### INDUSTRIAL APPLICATIONS

The constructs described herein are useful to quickly and accurately determine the solubility of a target protein in a cell. To practice this method, a cell containing a construct of this invention is cultured under conditions where the target protein is expressed and the expression of the reporter gene is inducible. As used herein, the term “inducible” shall mean that transcription of the reporter gene can be initiated in response to a specific stimulus. The specific stimulus that induces transcription of a protein solubility responsive promoter is insoluble protein in the cytoplasm of the cell. With cells of the Gram negative bacterium E. coli, for example, the cells should be grown in liquid medium rather than on agar plates for the reporter gene to be inducible.

Expression of the reporter gene is measured following expression of the target protein. This can be accomplished by measuring the amount of protein directly such as by measuring fluorescence of a fluorescent protein or by measuring the reporter protein by an immunoassay such as an ELISA assay. Alternatively, if the reporter gene is an enzyme, the amount of reporter produced can be measured using an assay that quantifies a product produced by enzymatic modification of a substrate compound, such as metabolism of X-gal or ONPG by the β-galactosidase enzyme. The amount of reporter protein produced will be directly proportional to the amount of insoluble target protein in the cytoplasm.

The quantity of insoluble protein in a specific sample can be determined by first preparing a standard curve correlating target protein insolubility with the level of reporter gene expression. This can be accomplished by culturing a host cell comprising the reporter construct together with a target expression construct and preparing a series of samples in which the various amounts of insoluble target protein are produced. Expression of the protein insolubility reporter is measured in each of these samples.

The amount of soluble and insoluble target protein can be measured quantitatively by lysing the host cells, separating soluble and insoluble material, for example by centrifugation or filtration, and measuring the amount of target protein in each fraction, for example by immunoassay such as ELISA or Western blot. Once a standard curve relating protein insolubility to reporter expression has been prepared, the amount of insoluble protein present in a test sample can be determined by measuring the expression of the protein insolubility reporter in that sample and calculating the amount of insoluble protein present from the standard curve.

The invention also provides a method of screening for mutations in a cell that improve the solubility of a protein. These methods involve treating a population of cells with a mutagen, and identifying those cells that exhibit an increase in expression of the target protein in soluble form. A “mutagen” is intended to include, but not be limited to chemical mutagens such as ethyl methane sulphonate, N-methyl-N′-nitroso-guanidine and nitrous acid as well as physical agents such as ionizing radiation. In an alternative embodiment, mutations can be introduced into a polynucleotide sequence encoding a target protein. The altered polynucleotide is then tested to determine whether the solubility of the target protein is changed. Such mutations include for example, mutations induced by a mutagen; site directed mutations that alter specific amino acid residues such as mutation of cysteine residues to eliminate disulfide bonds; deletions that remove sets of specific amino acids such as deletion of a continuous stretch of hydrophobic amino acids; and fusions of the target protein to a second, particularly soluble protein. In each case, the solubility of the target protein is assessed by determining expression of a protein solubility reporter nucleic acid as described herein.

To identify mutations that alter the solubility of a target protein, a polynucleotide that encodes this protein is expressed suitable conditions such that the reporter gene is responsive to expression of insoluble protein. If a mutation has been introduced that increases the solubility of the target protein then the level of expression of the reporter gene will be reduced as compared to the level of expression of the reporter gene observed in the host cell prior to treatment of this cell with the mutagen, provided that the protein solubility responsive promoter is upregulated in response to expression of insoluble protein. By selecting a reporter gene whose expression is easily measured in a large number of individual samples, such as the β-galactosidase gene, it is possible to use this method to screen a large number of independent mutations to identify alterations that improve the solubility of a target protein.

The constructs are also useful for identifying variations in a process for biosynthesis of a target protein. The process can be varied to modify the solubility of the target protein. A cell containing a protein solubility reporter nucleic acid is cultured under alternative conditions where the target protein is expressed and the reporter is inducible, and measuring the expression of the reporter gene, to identify variations in culture conditions that improve the solubility of the expressed target protein. For example, protein solubility may be affected by the temperature, medium composition, or oxygen concentration in which the cells are cultured. The convenient method by which expression of the reporter is measured allows a variety of alternative conditions to be tested with minimal effort, to identify those conditions where the highest proportion of soluble target protein is produced.

The constructs also are useful to compare alternative cells to identify a cell that synthesizes an increased amount of soluble target protein by performing a method identified herein with at least two alternative cells and comparing the amount of reporter gene expressed to identify a cell that expresses an increased amount of soluble target protein.

The present invention also provides a method of screening an expression library of clones to identify those clones that express soluble protein. This library can consist of alterations in the gene expressing the target protein of interest. Alterations of the gene can be provided by any of several widely used methods. These include making truncations in the gene, random chemical mutagenesis, random mutagenesis through erroneous nucleotide incorporation, or site-directed mutagenesis methods. This library of alterations is transformed into cells that contain the protein solubility reporter system. Individual clones of the transformed cells are then cultured under conditions where the target gene or its alterations are expressed. The level of reporter gene expression in each clone is measured during expression of target gene or its alterations. Clones expressing increased or reduced levels of the reporter gene are identified by measuring reporter gene levels of each clone and comparing to a clone expressing the unmodified target gene. Clones thus identified are expressing less insoluble protein and may contain more soluble derivatives of the target protein.

It will be apparent to individuals skilled in the art that selection of appropriate reporter genes for the protein solubility reporter system will enable the use of this system in a variety of efficient, high-throughput procedures to rapidly screen large number of alternative cultures in order to identify specific samples that produce soluble target proteins. The ease of detection of reporter genes such as β-galactosidase, luciferase, and green fluorescent protein further provides for the development of automated procedures to screen cells for target protein solubility.

In a further aspect, the constructs as defined herein are useful for identifying an antibiotic agent. The cells that contain the protein solubility reporter construct are contacted by a candidate agent. A potential antibiotic agent that interferes with the protein folding process will result in an increased expression of insoluble endogenous cellular protein, thereby inducing expression of a reporter gene that is under the control of a promoter that is upregulated in response to the presence of insoluble protein. Measurement of the reporter gene product is performed after treatment with the potential antibiotic agent. Cells expressing increased reporter activity relative to a control substance are an indication that the test agent is a potential antibiotic.

An additional aspect of the invention is to use the process described above with co-expression of a soluble protein which is a known target of antibiotic therapies. Agents that interfere with the folding of these known target proteins would result in insoluble protein and increased expression of the reporter gene. Agents thus identified would have potential utility as an antibiotic by interfering with the proper folding of these target proteins in their native hosts.

### EXAMPLES

The following examples are offered to illustrate, but not to limit the present invention.

Proper protein folding is key to producing recombinant proteins for structure determination. In this Example, the effect of misfolded recombinant protein on gene expression in E. coli is examined. Comparison of expression patterns indicates a unique set of genes responding to translational misfolding. The response is in part analogous to heat shock and suggests a translational component to the regulation. We have further utilized the expression information to generate reporters responsive to protein misfolding. These reporters were used to identify properly folded recombinant proteins and to create soluble domains of insoluble proteins for structural studies.

Materials and Methods

Cloning

Clones expressing properly folded or misfolded human proteins were obtained from the GeneStorm collection (Invitrogen). Clones containing the Unigene accession numbers L35545, U18291, M94856, M22146, D87116, M63167, M68520, M60527, M36881, M36981, U35003, S79522, X73460, D14520, U14968, M86400 were provided in the pBADThio vector (Invitrogen) to provide arabinose-inducible expression. T. maritima genes were amplified from genomic DNA and cloned into the expression vector pMH1 which encodes a 12 amino acid N-terminal tag containing a 6×-histidine repeat for purification and detection. Reporter vectors were constructed by inserting a PCR amplifer of 300 bp upstream of the ibpAB, ybeD, yhgI or yrjGHI genes upstream of beta-galactosidase in a pACYC184 derivative.

Rep 68 was cloned from a plasmid that contains the entire genome of the human adeno-associated virus 2 (AAV2). Putative domains comprised of bases 1-646, 647-1456, and 1457-1611 were amplified from the full-length template and cloned into pMH1. The above template was also used in amplifications of the full-length gene for fragmentation. Two μg of the rep 68 amplifer were used in each of 5 fragmentation reactions containing 1, 0.1, 0.01, 0.001, or 0 units of DNase I (Boeringer Mannheim) as well as Pfu polymerase and dNTPs. Reactions were set up on ice with the DNase added immediately prior to temperature cycling in an MJ Research thermocycler according to the following: 10 min@25° C., 15 min@95° C., and 30 min@72° C. Each reaction was run on a 1% agarose gel and fragments corresponding to 1600-1000 bp, 1000-850 bp, 850-600 bp, and 600-300 bp were extracted. Each pool was used as above for blunt cloning and ligation into pMH1 as above and introduced into the reporter cell line HK 57 for screening.

Cell Growth and Protein Expression

E. coli strains MG1655 (F lam rph1) and KY1429 (F-araD139 Δ(argF-lac)169 lam flhD5301 fruA25 relA1 rpsL150 zhh50::Tn10 rpoH606(ts) deoC1) were transformed with expression plasmids encoding M36881 (LCK) or M86400 (PLA) for expression profiling. Cells were cultured at 37° C. in Luria Broth containing ampicillin. Protein expression was induced by addition of L-arabinose to a final concentration of 0.1% for 1 hour. KY1429 cells were cultured as above except initial growth was performed at 32° C. followed by a shift to 42° C. for non-permissive expression of rpoH606. Top10 cells (F⁻mcrA Δ(mrr-hsdRMS-mcrBC) φ80lacZΔM15 ΔlacX74 deoR recA1 araD139 Δ(ara-leu) 7697 galU galK rpsL endA1 nupG) containing the ibpAB promoter fusion (pHK57), were transformed with expression constructs listed above. Beta-galactosidase assays were performed essentially as described by Miller (24). Fractionation of soluble and insoluble proteins was performed by centrifugation. Cultures were resuspended in 50 mM Tris pH 7.9, 50 mM NaCl, 1 mM MgCl₂, 3 mM methionine and sonicated for 2 minutes on ice. Cell debris and insoluble protein aggregates were pelleted by centrifugation at 3000×g for 15 minutes. The soluble fraction was removed and the pellets resuspended in an equivalent volume of lysis buffer.

Probe Preparation and Hybridization and Analysis of Labeled mRNA

Labeled mRNA was prepared and hybridized to an E. coli whole genome array (Affymetrix) essentially as described previously (25, 26). This gene chip contains 25-mer oligonucleotide probes for each of the 4290 known E. coli genes. Standard Affymetrix GeneChip analysis software was used to measure individual gene expression and to perform pairwise comparison of gene expression levels for pre-induction and post-induction samples. Comparisons of changes in gene expression for properly folded and misfolded genes were analyzed for individual gene probe sets.

Microplate Solubility Screening

Ninety-six well microplates containing 200 μL of LB with 100 μg/mL ampicillin and 34 μg/mL chloramphenicol were inoculated with single colonies from above and grown overnight with shaking at 37° C. Overnight cultures were used to inoculate 200 μL of the same media and incubated at 37° C. until reaching an average OD₆₀₀ of 0.5. Cultures were induced with a final concentration of 0.2% arabinose. After 30 minutes, a cocktail of cefatriaxone and cefotaxime was added to each well to a final concentration of 10 μg/mL of each and the plates were incubated for an additional 1.5 hours. Cultures were harvested after 2 hours total of induction by centrifugation at max speed for 15 minutes to pellet cell debris on the bottom of the wells. The soluble lysate was then separated 25 μL into one set of clean microplates for β-galactosidase activity screens and 75 μL into Nunc Maxisorp™ ELISA plates for Ni-HRP screening.

β-galactosidase activity screening of lysates was performed using a variation of the Miller protocol (10). 50 μL of 4×Z-buffer and 50 μL of 4×ONPG were added to microplates containing 25 μL of soluble lysate. After development of yellow color in positive control wells, the reaction was quenched with 75 μL of 1M Na₂CO₃ pH 8. The A₄₂₀, A₅₅₀ and reaction times were recorded and used along with the OD₆₀₀ data to calculate β-galactosidase activity (10).

Ni-HRP screening was performed similar to an ELISA. 75 μL of lysate plus 25 μL TBS was bound overnight at 4° C. to a microtiter plate and blocked with 1% (w/v) BSA in TBS for 4 hours at 25° C. Plates were then washed 3×with TBST, 100 μL of Ni-HRP conjugate (KPL Labs) was added at a dilution of 1:2500 and incubated 1 hour at 25° C. The plates were then washed with TBST and 100 μL of the HRP substrate (KPL Labs) was added and color was allowed to develop until the positive control well was deep blue. The reaction was quenched with 100 μL 1N HCl and the A₄₂₀ determined. Solubility scores were calculated by weighting the Ni-HRP A₄₂₀ readings such that the mean was one order of magnitude greater than the mean of the β-galactosidase activity scores and dividing the Ni-HRP absorbance by the β-galactosidase activity.

Results

Analysis of Gene Expression

To examine gene expression as a result of misfolded protein, representative genes were cloned as fusion proteins to thioredoxin under control of the tightly regulated arabinose promoter. Human phospholipase A2 (PLA) is almost entirely soluble, as determined by cell lysis and fractionation by centrifugation. Further evidence of proper folding of this protein was obtained through dynamic light scattering of purified protein and the ability to crystallize it from a single affinity purification step. Under equivalent expression conditions, human lymphocyte-specific protein tyrosine kinase (LCK) is expressed almost exclusively as insoluble protein. Both proteins were expressed at sufficient levels to be the predominant translation product. mRNA preparations from induced and non-induced cultures were prepared and used to probe for gene expression.

Recombinant protein expression within E. coli is predicted to cause a substantial change in gene expression. Indeed, a comparison of gene expression with a pre-induction control shows 6% of total genes showing>3-fold differences in expression in both cases. In the case of insoluble recombinant protein, 27 genes show>10-fold changes in expression, as compared to 10 genes in the case of the soluble recombinant protein. A comparison of the two profiles identifies 53 genes listed in Table 1 showing>3-fold changes, that are unique to the insoluble case. These genes, then, are likely responsive to misfolded protein in the cell and may play a role within E. coli in dealing with this translational stress.

The heat shock transcription factor RpoH is normally repressed by interaction with the chaperone protein DnaK. In the presence of misfolded protein, DnaK binds to that protein thereby allowing RpoH to stimulate transcription of heat shock promoters (7). Upstream regions of many of the induced genes in Table 1 show the presence of RpoH-dependant promoter sequences. Further evidence of the important role played by RpoH is provided by expression profiling results performed from an rpoH606 mutant (KY1429) expressing misfolded LCK protein compared to a non-expressing control. A strikingly different expression profile is seen in the case of the rpoH606 mutant (Tables 1 & 2). The majority of the genes induced by the misfolded protein in the wild-type strain are poorly induced in the rpoH606 mutant indicating that they are directly or indirectly under control of this transcription factor.

Induction of Heatshock Genes

Not surprisingly, many of the genes induced by translational misfolding have known chaperone activity. These include the well-characterized dnaJ, dnaK, and grpE genes. The corresponding proteins interact as a complex with misfolded or denatured protein in an ATP-dependant repair process. Likewise, mopAB genes forming the GroELS folding repair complex are induced under translational misfolding conditions. IbpAB are small heatshock polypeptides associated with inclusion body aggregates of recombinant protein (13). While they do not appear to behave as folding chaperones directly, they bind misfolded protein and interact with the DnaJK GrpE proteins as a chaperone system (14). Hsp33, the gene product of the yrfI gene was recently identified as a chaperone protein responsive to oxidizing conditions (15). Genes implicated in degradation of denatured protein are also induced by translational misfolding. The Ion, clpBP, and hslUV protease genes are expressed at increased levels. Under normal cell growth these proteases serve an important recycling function. Insoluble aggregates are relatively resistant to proteolysis and this recycling pathway is ineffective for recombinant protein expression.

TABLE 1
Fold change in gene expression for genes unique to misfolded response.
Fold
Increase

| | | | | | |
|---|---|---|---|---|---|
| Heat | | | | | |
| shock⁽⁹⁾ | Misfolded | Folded | Control | rpoH(−) | function |

Heat shock gene
(SEQ ID NO:)

| | | | | | | |
|---|---|---|---|---|---|---|
| ibpA (21) | 297.4 | 74.4 | −1.7 | −1.5 | 14.3 | chaperone |
| ibpB | 327.2 | 40.0 | 2.7 | 1.4 | 10.4 | chaperone |
| yrfH | 51.3 | 28.3 | −3.2 | 1.6 | 2.8 | ribosome associated HSP |
| yccV (19) | 34.3 | 19.3 | −2.4 | −1.9 | 3.8 | unknown |
| fxsA (14) | 50.7 | 22.3 | 2.1 | −3.8 | 2.0 | suppresses F exclusion of |
| | | | | | | phage T7 |
| dnaK (18) | 58.5 | 16.6 | 3.2 | 1.8 | 3.8 | chaperone |
| htpG (17) | 33.8 | 13.2 | −2.6 | −3.5 | 3.0 | chaperone |
| clpP (16) | 3.3 | 11.8 | −3.6 | 2.6 | 2.7 | protease |
| yhdN (22) | 9.5 | 11.1 | 3.9 | 1.5 | 3.9 | unknown |
| clpB (13) | 36.5 | 9.7 | −1.3 | 2.3 | 7.0 | protease |
| hslV (15) | 16.2 | 7.4 | 1.7 | 1.1 | 3.5 | protease |
| mopA | 37.9 | 6.4 | −1.2 | 1.9 | 1.7 | chaperone |
| lon | 20.3 | 6.0 | 1.3 | −1.0 | 2.9 | protease |
| mopB | 77.5 | 5.8 | 1.1 | 2.4 | 1.7 | chaperone |
| dnaJ | 85.3 | 5.6 | 2.7 | 1.2 | 4.2 | chaperone |
| yrJG (20) | 12.1 | 5.0 | −1.3 | −1.0 | 2.1 | unknown |
| htpX (12) | 36.1 | 4.9 | −1.3 | −2.5 | 2.6 | HSP, unknown |
| hslU | 10.3 | 4.7 | −2.1 | −1.0 | 3.0 | protease |
| grpE (11) | 24.1 | 3.9 | 1.5 | −1.4 | 2.6 | chaperone |
| yrfI | 21.6 | 3.6 | −1.1 | −1.5 | 2.7 | chaperone |
| rrmJ | 9.1 | 3.2 | 1.5 | −1.2 | 3.0 | rRNA methylase |

Other induced

| | | | | | |
|---|---|---|---|---|---|
| yagU (9) | 17.4 | 2.4 | 2.6 | 3.1 | unknown |
| yciS (8) | 14.8 | 2.6 | 1.8 | 5.4 | unknown |
| ybeD (7) | 12.0 | 3.8 | 1.3 | 1.8 | unknown |
| araE | 11.6 | 3.1 | 1.2 | 17.7 | arabinose transport |
| yojH (6) | 9.7 | 5.2 | −1.9 | −3.0 | unknown |
| yejG (5) | 7.3 | 3.0 | 1.6 | 5.0 | unknown |
| exbB (4) | 6.4 | −4.3 | −1.6 | 1.7 | uptake of enterchelin |
| yhgI | 5.3 | −1.0 | 1.0 | 2.2 | unknown |
| proP (3) | 5 | −1.0 | 1.0 | 5.1 | proline transport |
| kgtP (1) | 4.2 | 2.4 | −1.2 | 3.2 | alpha-ketoglutarate |
| | | | | | permease |

Downregulated

| | | | | | |
|---|---|---|---|---|---|
| recR | −17.9 | −9.0 | −3.3 | −3.0 | recombination and DNA repair |
| lamB | −9.9 | −4.8 | 12.4 | −5.7 | maltose uptake |
| glpD | −9 | −2.3 | −2.9 | −8.1 | glycerol-3-phosphate |
| | | | | | dehydrogenase |
| yfiD | −8.6 | −3.2 | 5.3 | −1.4 | unknown |
| rbsC | −8.2 | −5.9 | 3.3 | −3.0 | D-ribose transport |
| glpF | −8 | −3.2 | −3.9 | −10.3 | glycerol facilitator |
| yqjE | −7.7 | −7.6 | −1 | 1.1 | unknown function |
| ftsZ | −7.2 | −8.0 | −1.4 | 1.6 | cell division; initiation of |
| | | | | | septation |
| ycfN | −7.1 | −1.3 | −1.5 | 1.3 | unknown function |
| feoA | −7.1 | −4.3 | 2.1 | 1.1 | ferrous iron uptake |
| ybjC | −6.9 | −5.2 | −1.5 | −2.4 | unknown function |
| yccA | −6.9 | −5.0 | −3.6 | −1.2 | unknown function |
| deoA | −6.9 | −4.3 | 2.1 | −1.7 | thymidine phosphorylase |
| deoB | −6.9 | −4.2 | −1.3 | −3.6 | deoxyribouratase, |
| | | | | | phosphopentomutase |
| nrdB | −6.7 | −2 | −2.2 | −3.2 | ribonucleoside diphosphate |
| | | | | | reductase |
| fecB | −6.7 | −4.5 | −2.2 | −2.6 | citrate-dependent iron |
| | | | | | transport |
| ycaR | −6.5 | −1.2 | −1.7 | −2.4 | unknown |
| tnaL | −6.1 | −4.2 | 22.3 | 1.1 | regulatory leader for tna |
| | | | | | operon |
| speD | −5.8 | −2 | −2.1 | −3.5 | S-adenosylmethionine |
| | | | | | decarboxylase |
| rfbD | −5.8 | 1.1 | −3.8 | −1.2 | TDP-rhamnose synthetase |
| ybaB | −5 | −2.2 | −2.2 | −6.0 | unknown function |

TABLE 2
Fold increase in cold shock protein (csp) gene expression
after induction of misfolded protein expression

| | rpoH(+) | rpoH(−) |
|---|---|---|
| cspB | 2.6 | 142.6 |
| cspG | 8.1 | 50.1 |
| cspA | 3.5 | 4.3 |
| cspI | 1.9 | 9.8 |

Induction of Ribosome Associated Genes

Other heat shock genes associated with the ribosome are induced under conditions of translational misfolding. Hsp15 (yrfH) binds RNA (24) and is associated with free 50S ribosomal subunits containing a nascent polypeptide chain (16). Heat-shock also increases the level of Hsp15-binding implying increased dissociation of 50S and 30S subunits. Further suggestion of ribosomal dissociation comes from the induction of ftsJ (rrmJ) (SEQ ID NO:10). The ftsJ gene product is an RNA methylase specific for 23S rRNA only when contained in the 50S ribosomal subunit (17, 18). This enzyme methylates 23S rRNA at position 2552 located within the peptidyl transferase center of the ribosome (17). Mutants in ftsJ lack methylation of 23S rRNA and show up to 65% decrease in ribosomal activity corresponding to dissociation of the 50S and 30S subunits (19). Particularly striking in the rpoH mutant is the large increase in transcripts of the cold-shock proteins (CSPs) (Table 2). These genes were not affected by heat-shock (9), but are associated with a transient halt of translation. CSPs are RNA binding proteins which act as chaperones for untranslated message (20, 21) and provide anti-termination activity (22). Increased expression of CSPs under conditions which reduce chaperone expression (rpoH606) is an indication of paused translation. Taken together, these results suggest a translational regulatory response, possibly as a result of demethylation, as a consequence of translational misfolding. This hypothesis is an interesting regulatory mechanism currently under investigation.

Other Induced Genes

yccV, yhdN, and yrfG have been shown to increase expression under heat shock conditions but are of unknown function. In addition to these known heats shock genes, yagU, yciS, ybeD, yejG, and yhgI show increased expression. Most of these proteins are relatively small and generally acidic. One speculation is that some of these proteins perform a similar role to IbpAB in the direct recognition and sequestering of misfolded protein. However, only IbpAB have been associated with misfolded and aggregated protein. Induction levels of ibpAB are much higher and these other proteins may be present at lower levels. Interestingly, knockout mutations of ibpAB have relatively little affect on cell growth and viability (14) suggesting some functional redundancy within the cell.

Genetic Reporter of Protein Folding

To confirm the profiling results and facilitate experimentation with a larger number of recombinant proteins, we cloned the promoter regions from ibpAB, ybeD, yhgI and yrfGHI into a beta-galactosidase reporter vector. In each case, increased beta-galactosidase activity was observed when expression of the misfolded protein LCK was induced whereas the folded protein PLA showed no increase in activity. These results were further extended using a set of 8 misfolded proteins and 6 properly folded proteins co-expressed in the presence of the ibpAB-promoter beta-galactosidase fusion. In each case, increased beta-galactosidase activity corresponded to expression of misfolded protein. A more detailed characterization is shown below. The response observed, then, appears to be a general result of protein misfolding rather than a specific response to any particular protein. These reporters provide a simple means of identifying misfolded protein through a sensitive enzymatic assay and the ibpAB promoter fusion was chosen as the reporter for further studies.

ELISA-Like Assay for Soluble Protein

For identifying protein derivatives that have improved folding properties in a recombinant environment, we also developed an ELISA-like assay compatible with high-throughput screening instrumentation. To evaluate soluble protein levels in a high-throughput system, non-denatured cell lysates must be prepared using conditions compatible with rapid screening in microplates. In lieu of the detergent or organic lysis, we added an antibiotic cocktail to each well to induce lysis. Soluble protein fractions were removed, bound to microtiter plates, and recombinant protein detected via binding of a Ni-HRP conjugate to a 6×-histidine N-terminal fusion. It should be noted that the His-tag may not be uniformly accessible among recombinant proteins. A negative Ni-HRP response, therefore, may not be indicative of an absence of soluble protein, but the protein fold may occlude access to the His-tag. However, we have not observed this to be a common problem. This assay, then, provides a measure of the levels of soluble recombinant protein without the need to run an SDS gel and in a form that is compatible with a HT-screen and the β-galactosidase assay.

Testing Proteins with Pre-Determined Expression Characteristics

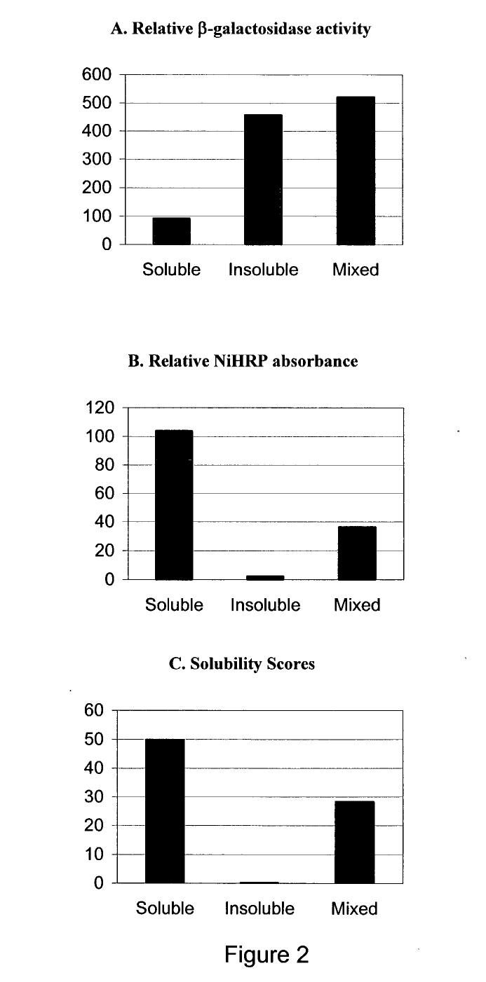

As part of our effort aimed at cloning, expressing, and characterizing the total proteome of Thermotoga maritima, we tested the efficacy of the reporter on a set of 18 T. maritima proteins shown in Table 3 (6 soluble, 6 insoluble, and 6 mixed solubility). To optimize assay parameters, strains were arrayed in 96-well plates and assayed in triplicate at three induction levels (0.02%, 0.2%, and 2% arabinose) and at four post-induction time points for addition of the lysis-promoting antibiotics (t=0 min, 30 min, 60 min, and 120 min after addition of arabinose). FIG. 2 shows the averaged results for triplicate plates (soluble, insoluble and mixed) for the 0.2% arabinose induction. Both the insoluble and the mixed pools showed greater than four-fold higher β-galactosidase activity than the soluble pool (FIG. 2A). Conversely, the soluble pool showed a greater than ten-fold higher response in the Ni-HRP assay opposed to the insoluble pool (FIG. 2B). The mixed pool, comprised of proteins expressed approximately equally in both soluble and insoluble fractions, showed Ni-HRP binding approximately half the intensity of the soluble pool. Although either lack of β-galactosidase or presence of Ni-HRP activity alone could be used as a measure of soluble protein, we chose a ratio of the two activities a more effective and convenient screen.

TABLE 3
T. maritima proteins with pre-determined expression characteristics

| | | | |
|---|---|---|---|
| Accession # | ID | MW | pI |

Soluble Expression

| | | | |
|---|---|---|---|
| TM0560 | conserved hypothetical protein | 20.62 | 5.34 |
| TM0414 | dehydrogenase | 37.485 | 5.49 |
| TM0574 | S-adenosylmethionine tRNA | 38.662 | 8.61 |
| | ribosyltransferase (queA) | | |
| TM0703 | competence-damage inducible protein, | 45.181 | 6.49 |
| | putative | | |
| TM0554 | 3-isopropylmalate dehydratase, large | 45.286 | 5.92 |
| | subunit (leuC) | | |
| TM0556 | 3-isopropylmalate dehydrogenase (leuB) | 39.19 | 5.44 |

Insoluble Expression

| | | | |
|---|---|---|---|
| TM0688 | glyceraldehyde-3-phosphate | 36.425 | 6.21 |
| | dehydrogenase (gap) | | |
| TM0633 | flagellar-related protein | 15.826 | 5.96 |
| TM0712 | conserved hypothetical protein | 28.28 | 5.69 |
| TM0343 | chorismate mutase, putative | 37.378 | 6.22 |
| TM0218 | flagellum-specific ATP synthase (fliI) | 48.326 | 6.15 |
| TM0294 | glutamate 5-kinase (proB) | 38.32 | 6.92 |

Mixed Soluble/Insoluble Expression

| | | | |
|---|---|---|---|
| TM0289 | 6-phosphofruktokinase, pyrophosphate- | 46.465 | 6.55 |
| | dependent | | |
| TM0564 | conserved hypothetical protein | 18.625 | 5.86 |
| TM0540 | fumarate hydratase, N-terminal subunit | 30.554 | 6.18 |
| TM0425 | oxidoreductase, putative | 37.403 | 6.62 |
| TM0731 | conserved hypothetical protein | 22.509 | 9.27 |
| TM0413 | creatinine amidohydrolase, putative | 34.947 | 5.57 |

Testing Proteins with Unknown Expression Characteristics

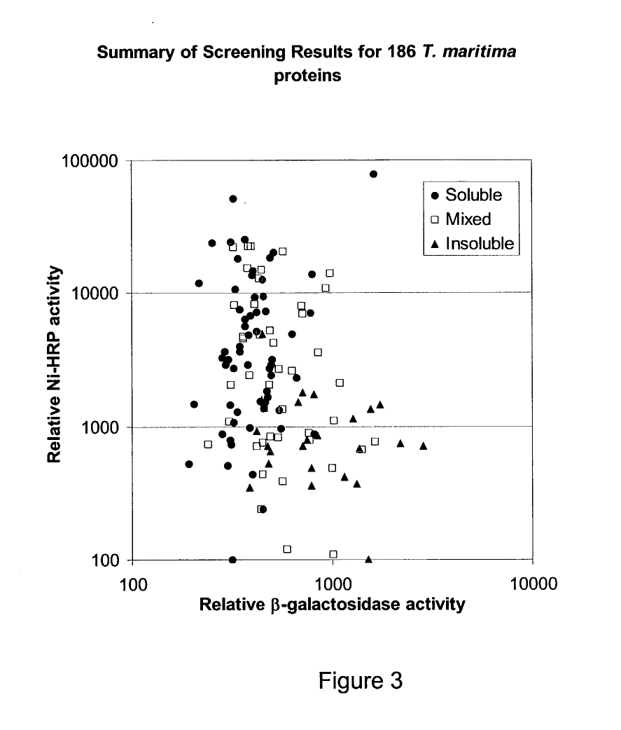

We next applied this screen to a large set of proteins with unknown folding properties. We performed the screen under the optimal conditions noted above on 186 T. maritima proteins not previously characterized for expression. The results of this screen are summarized in Table 4. SDS-PAGE of eluates from nickel-chelating resin and the dissolved insoluble fractions for each clone was performed along with corresponding β-galactosidase activity, Ni-HRP response, and solubility scores for 186 clones. Based on the results of the gels, 57 clones did not overexpress a visible protein band, 62 clones expressed predominantly soluble protein, 27 expressed predominantly insoluble aggregates, and 46 expressed approximately equally to both soluble and insoluble fractions. A comparison of β-galactosidase activity to Ni-HRP assay is shown in FIG. 3. Points are categorized by SDS gel analysis of the soluble and insoluble protein fractions. The screen positively identified 54 of 62 (87%) soluble proteins. Seven of the eight remaining proteins that were soluble according to the gels had low Ni-HRP assays, most likely due to inaccessibility of the His-tag in these fusion proteins. Taken alone, the β-galactosidase activity measurement identified 22 of 27 (81%) insoluble proteins. Those proteins showing partial solubility showed variable solubility scores, suggesting partial folding is inducing β-galactosidase through the reporter. This assay, then, provides an effective and convenient means of classifying folding characteristics.

TABLE 4
Average solubility screen values for T. maritima proteins

| | |
|---|---|
| 186 T. maritima proteins | Rep 68 |

| | Soluble | Insoluble | Mixed | Screen | Domain |
|---|---|---|---|---|---|
| Relative β-galacto- | 96.1 | 681 | 283 | 284 | 3.1 |
| sidase activity | | | | | |
| Relative NiHRP | 776 | 94.6 | 525 | 561 | 1700 |
| Absorbance | | | | | |
| Solubility Score | 297 | 2.94 | 83.1 | 3.7 | 552 |

Identification of Soluble Protein Domains

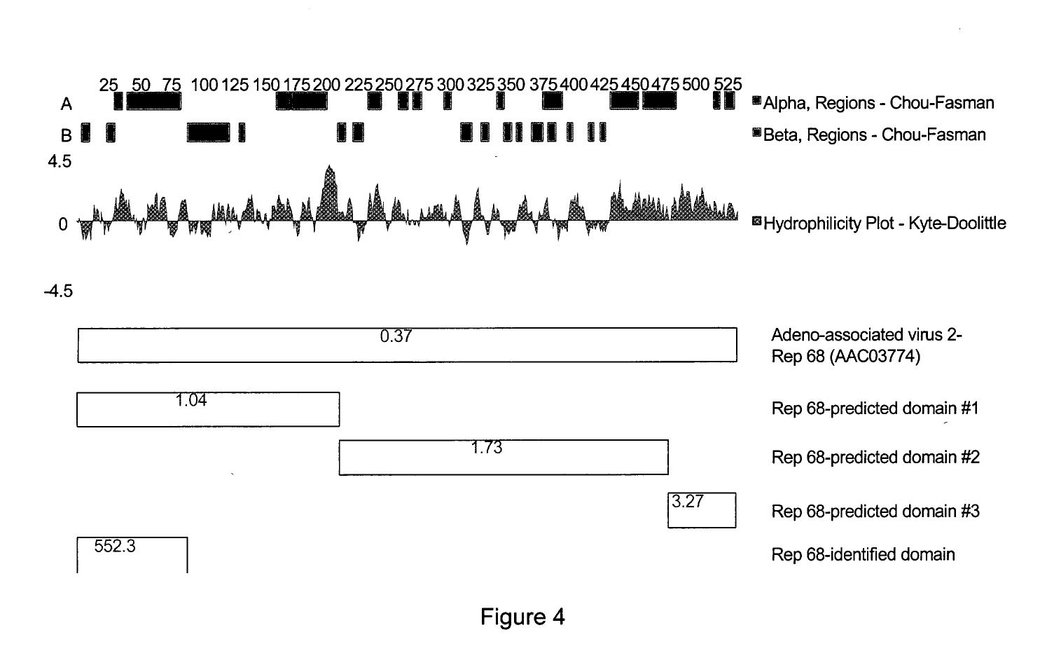

One utility of this system lies in the ability to identify variants of full-length gene products, either mutants or domains, based on improved properties. For structural and biochemical studies, we tested the ability of this screen to identify soluble fragments of Rep68 (GI: 209617), an adeno-associated virus non-structural protein possessing various activities related to the integration of the viral genome into target DNA. This protein previously had been found to express predominantly as unfolded aggregates in E. coli. We performed both a random approach and a rational approach based on selection of domains with regard to homology. Three domains of Rep68 were selected after an RPS-BLAST search (http://www.ncbi.nlm.nih.gov/Structure/cdd/wrpsb.cgi) identified an internal domain possessing homology to a parvovirus non-structural protein, NP-1. This information, combined with a Kyte-Doolittle hydropathy plot (FIG. 4), was used to assign the 5′ and 3′ cutoffs for each domain. The remaining N-terminal and C-terminal residues comprised the other two domains and did not possess significant homology to any other proteins in the database. Random fragments of Rep68 were also generated for screening by DNase fragmentation.

The three predicted domains and randomly generated fragments were screened response to identify soluble fragments of Rep68. None of the three predicted domains were identified as soluble (FIG. 4) Concurrently, 564 randomly generated fragments of rep68 were also screened. One fragment returned a significantly high solubility score (Table 4). This clone was verified by large-scale expression and showed expression in both the soluble and insoluble fractions. Subsequent sequencing of the identified clone verified that it was comprised of a fragment of rep68 corresponding to amino acids 1-95 (FIG. 4). The identified fragment showed substantial improvement in solubility over the full-length protein and is being tested in crystallization trials.

Conclusion

Most gene expression studies of in vivo protein folding have focused on denaturation as a result of environmental stress. This response is essential in vivo to deal with ever-changing environmental and non-ideal growth conditions. Translational folding issues are equally important to the cell since every protein as it is being translated is essentially in an unfolded state. Expression of unnatural proteins, either through recombinant means or mutation is a “stress” in itself. We show that the cellular response to translational misfolding, like heat shock, involves many known chaperone genes with a clear inference how these gene products are involved in the folding of the nascent polypeptide. In addition, other non-heat shock genes and genes of unknown function are induced. These too may be involved in the folding process. Our results suggest both transcriptional and translational regulation. The DnaK-RpoH interaction is well-characterized and appears to be the major regulator of the transcriptional response. The altered expression of genes implicated in translational stalling and ribosomal dissociation is intriguing and implies that these effects might be a result of translationally misfolded protein. These genes include yrfH which associates with the 50S ribosomal subunit (16). cspABGI are induced in rpoH606 suggesting translational stalling in the absence of induced chaperones. Also included is ftsJ, which is known to methylate the 23S rRNA of 50S subunits resulting in higher affinity of the two subunits for each other (19). Ribosome structure shows that the location of the methylation site of ftsJ, position 2552 of the 23S rRNA, is intriguingly close to the peptidyl transferase center (18) making it an obvious potential regulator mechanism for a ribosomal sensor of misfolded protein. Such a ribosomal sensor is not unprecedented as demonstrated by the well-characterized stringent response to uncharged tRNAs during translation (23). Ribosomal stalling provides a mechanism to allow time for chaperone synthesis and recruitment thereby preventing irreversible aggregation. In this way, the cell would retain an additional salvage pathway where the emerging protein was held in the relatively protected environment of the translating ribosome until sufficient chaperones could be recruited.

The differentially regulated genes identified provide a valuable opportunity to create novel reporters of the folding state of cellular proteins as a whole and over-expressed, recombinant proteins in particular. Our reporter assay differs from others recently described by not relying on direct coupling of the reporter gene to the target, thereby limiting potential interference by the reporter. The combination of the Ni-HRP and β-galactosidase assays provides an effective means of assaying soluble recombinant proteins in a high-throughput way. We have extended this system to identify mutants and truncations of single gene products as a strategy to identify soluble domains of otherwise misfolded, aggregated proteins. Using this approach, we have identified soluble fragments of Rep68 and anticipate that this assay will provide a general means of isolating recombinant protein suitable for structure/function work.

### REFERENCES

1. Sauer, R. T. & Parsell, D. A. (1989) Genes Dev. 3, 1226-1232.

2. Mogk, A., Tomoyasu, T., Goloubinoff, P., Rudiger, S., Roder, D. Langen, H. & Bukau, B. (1999) EMBO J. 18, 6934-6949.

3. Beckmann, R. P., Mizzen, L. A. & Welch, W. J. (1990) Science 248, 850-854.

4. Hartl, F. U. (1996) Nature (London) 381, 571-580.

5. Gething, M. -J. (1997) Nature (London) 388, 329-331.

6. Goloubinoff, P., Mogk, A., Zui, A. P. B., Tomoyasu, T. & Bukau, B. (1999) Proc. Natl. Acad. Sci. USA 96, 12732-12737.

7. Liberek, K. & Georgopoulos, C. (1993) Proc. Natl. Acad. Sci. USA 90, 11019-11023.

8. McCarty, J. S., Rudiger, S., Schonfeld, H. -J., Schneider-Mergener, J., Nakahigashi, K., Yura, T., & Bukau, B. (1996) J. Mol. Biol. 256, 829-837.

9. Richmond, C. S., Glasner, J. D., Mau, R., Hongfan, J. & Blattner, F. R. (1999) Nucleic Acids Res. 27, 3821-3835.

10. Maxwell, K. L., Mittermaier, A. K., Forman-Kay, J. D., & Davidson, A. R. (1999) Protein Sci. 8, 1908-1911.

11. Waldo, G. S., Standish, B. M., Berendzen, J., & Terwilliger, T. C. (1999) Nat. Biotech. 17, 691-695.

12. Wigley, W. C., Stidham, R. D., Smith, N. M., Hunt, J. F., & Thomas, P. J. (2001) Nat. Biotech. 19, 131-135.

13. Allen, S. P., Polazzi, J. 01, Gierse, J. K. & Easton, A. M. (1992) J. Bacteriol. 174, 6938-6947.

14. Thomas, J. G. & Baneyx, F. (1998) J. Bacteriol. 180, 5165-5172.

15. Veinger, L., Diamant, S., Buchner, J. & Goloubinoff, P. (1998) J. Biol. Chem. 273, 11032-11037.

16. Korber, P., Stahl, J. M., Nierhaus, K. H. & Bardwell, J. C. A. (2000) EMBO J. 19, 741-748.

17. Caldas, T., Binet, E., Bouloc, P., Costa, A., Gesgres, J. & Richarme, G. (2000) J. Biol. Chem. 275, 16414-16419.

18. Puglisi, J. D., Blanchard, S. C. & Green, R. (2000) Nat. Struct. Biol. 7, 855-861.

19. Caldas, T., Binet, E., Bouloc, P. & Richarme, G. (2000) Biochem. Biophys. Res. Comm. 271, 714-718.

20. Wang, N., Yamanake, K. & Inouye, M. (1999) J. Bacteriol. 181, 1603-1609.

21. Jing, W., Hou, Y. & Inouye, M. (1997) J. Biol. Chem. 272, 196-202.

22. Bae, W., Xia, B., Inuoye, M. & Severinov, K. (2000) Proc. Natl. Acad. Sci. USA 97, 7784-7789.

23. Cashel, M., Gentry, D. R., Hernandez, V. J. & Vinella, D. (1996) The stringent response. in Escherichia coli and Salmonella Cellular and Molecular Biology, ed. Neidhardt, F. C. (ASM Press, Washington D.C.) pp. 1458-1496.

24. in Molecular Cloning a Laboratory Manual (1989) eds. Sambrook, J., Fritsch, E. F. & Maniatis, T., (Cold Spring Harbor Laboratory Press), p. 1735.

25. Lockhart, D. J., Dong, H., Byrne, M. C., Follettie, M. T., Gallo, M. V., Chee, M. S., Mittmann, M., Wang, C., Kobayashi, M., Horton, H. & Brown, E. L. (1996) Nat. Biotechnol. 14, 1675-1680.

26. Wodicka, L., Dong, H., Mittmann, M., Ho, M. H., & Lockhart, D. J. (1997) Nat. Biotechnol. 15, 1359-1367.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference for all purposes.

We claim:

1. A host cell that comprises:
a) a solubility reporter nucleic acid that comprises a protein solubility responsive promoter operably linked to a reporter gene; and
b) a target polypeptide-expressing nucleic acid that comprises a polynucleotide that encodes a target polypeptide;
wherein expression of the target polypeptide in an insoluble form causes a change in expression of the reporter gene.

2. The host cell of claim 1, wherein the solubility responsive promoter comprises a polynucleotide sequence that is at least 75% identical to a polynucleotide selected from the group consisting of SEQ ID NOS:1-22.

3. The host cell of claim 2, wherein the solubility responsive promoter comprises a polynucleotide selected from the group consisting of SEQ ID NOS:1-22.

4. The host cell of claim 1, wherein the solubility responsive promoter comprises a polynucleotide that comprises a regulatory region of a gene listed in Table 1.

5. The host cell of claim 1, wherein the solubility responsive promoter comprises a polynucleotide that comprises an RpoH recognition site.

6. The host cell of claim 5, wherein the solubility responsive promoter comprises a polynucleotide that is at least 75% identical to a polynucleotide selected from the group consisting of SEQ ID NOS:23-43.

7. The host cell of claim 6, wherein the solubility responsive promoter comprises a polynucleotide selected from the group consisting of SEQ ID NOS:23-43.

8. The host cell of claim 1, wherein the solubility responsive promoter is upregulated when the target polypeptide is expressed in insoluble form.

9. The host cell of claim 1, wherein the solubility responsive promoter is downregulated when the target polypeptide is expressed in insoluble form.

10. The host cell of claim 1, wherein the polynucleotide that encodes the target polypeptide is heterologous to the host cell.

11. The host cell of claim 1, wherein the target protein-expressing nucleic acid comprises a promoter operably linked to the polynucleotide that encodes the target polypeptide.

12. The host cell of claim 11, wherein the target protein-expressing nucleic acid comprises a promoter that is heterologous to the host cell.

13. The host cell of claim 11, wherein the target protein-expressing nucleic acid comprises a promoter that is heterologous to the polynucleotide that encodes the target polypeptide.

14. The host cell of claim 1, wherein the protein solubility responsive promoter is a prokaryotic promoter.

15. The host cell of claim 14, wherein the protein solubility responsive promoter is a Gram negative bacterial promoter.

16. The host cell of claim 15, wherein the Gram negative bacterium is a member of the family Enterobacteriaceae.

17. The host cell of claim 16, wherein the member of the family Enterobacteriaceae is selected from the group consisting of the genera Escherichia, Salmonella, Shigella, Klebsiella and Enterobacter.

18. The host cell of claim 17, wherein the Gram negative bacterium is E. coli.

19. The host cell of claim 14, wherein the protein solubility responsive promoter is a Gram positive bacterial promoter.

20. The host cell of claim 1, wherein the protein solubility responsive promoter is a eukaryotic promoter.

21. The host cell of claim 20, wherein the promoter is a mammalian, plant, insect, fungal, or yeast promoter.

22. The host cell of claim 1, wherein the reporter gene comprises a polynucleotide that encodes a selectable or detectable polypeptide.

23. The host cell of claim 22, wherein the selectable or detectable polypeptide is selected from the group consisting: a metabolic enzyme, antibiotic resistance factor, a chemiluminescent protein, and a fluorescent protein.

24. The host cell of claim 23, wherein the detectable polypeptide is β-galactosidase.

25. The host cell of claim 23, wherein the detectable polypeptide is a luminescent or fluorescent protein.

26. The host cell of claim 22, wherein the reporter gene further comprises a polynucleotide that encodes a signal peptide that directs the detectable polypeptide to a surface of the host cell.

27. The host cell of claim 26, wherein the reporter gene further comprises a molecular tag that facilitates separation of a host cell that expresses the reporter gene from a host cell that does not express the reporter gene.

28. The host cell of claim 1, wherein the protein solubility responsive promoter is from the same species as is the host cell.

29. The host cell of claim 1, wherein the target polypeptide comprises a fragment of a larger polypeptide.

30. The host cell of claim 29, wherein the fragment comprises a domain of the larger polypeptide.

31. The host cell of claim 30, wherein the domain is identified by homology to other polypeptides, by hydropathy plot, or both.

32. The host cell of claim 29, wherein the fragment comprises a polypeptide encoded by a random fragment of a polynucleotide that encodes the larger polypeptide.

33. The host cell of claim 1, wherein the target polypeptide comprises a mutated form of a polypeptide.

34. An array of two or more populations of host cells of claim 1, wherein the host cells of each population differ in the target polypeptides expressed by the host cells.

35. The array of claim 34, wherein the polypeptides differ due to amino acid substitutions, deletions, or insertions compared to a reference amino acid sequence.

36. The array of claim 34, wherein the target polypeptides expressed by the populations of host cells comprise different fragments of a larger polypeptide.

37. A method of determining the solubility of a target polypeptide, the method comprising:
a) culturing a host cell of claim 1 under conditions in which the target polypeptide is expressed; and
b) determining whether expression of the reporter gene is increased or decreased, thereby determining the solubility of the expressed target polypeptide.

38. The method of claim 37, wherein the host cell is a prokaryotic cell.

39. The method of claim 38, wherein the host cell is an E. coli cell.

40. The method of claim 37, wherein the solubility responsive promoter comprises a polynucleotide sequence that is at least 75% identical to a polynucleotide sequence selected from the group consisting of SEQ ID NOS:1-43.

41. The method of claim 40, wherein the solubility responsive promoter comprises a polynucleotide sequence selected from the group consisting of SEQ ID NOS:1-43.

42. The method of claim 37, wherein the host cell is a eukaryotic cell.

43. The method of claim 37, wherein expression of the reporter gene is determined by performing a quantitative assay to determine the amount of detectable or selectable polypeptide in the cell.

44. The method of claim 37, wherein the host cells are subjected to cell sorting to separate cells having increased or decreased expression of the reporter gene from cells in which expression of the target polypeptide does not change the expression level of the reporter gene.

45. The method of claim 44, wherein the reporter gene encodes a fluorescent protein and the cell sorting comprises fluorescence activated cell sorting.

46. The method of claim 37, wherein:
the solubility reporter nucleic acid further comprises:
a) a polynucleotide that encodes a molecular tag; and
b) a polynucleotide that encodes a signal peptide;
wherein the signal polypeptide, the molecular tag, and a detectable or selectable polypeptide encoded by the reporter gene are expressed as a fusion protein and the signal polypeptide directs the detectable or selectable polypeptide to a surface of the cell;
and the method further comprises contacting host cells with a solid support to which the molecular tag can bind, wherein cells that express the reporter gene are immobilized on the solid support.

47. The method of claim 46, wherein the solubility responsive promoter is downregulated when the target polypeptide is expressed in insoluble form, and host cells that express the target polypeptide in insoluble form do not bind to the solid support.

48. The method of claim 46, wherein the solubility responsive promoter is upregulated when the target polypeptide is expressed in insoluble form, and host cells that express the target polypeptide in insoluble form bind to the solid support.

49. The method of claim 46, wherein the molecular tag comprises an epitope for an antibody, a poly-histidine tag, or a FLAG™ peptide.

50. The method of claim 37, wherein the method further comprises:
lysing the host cells under nondenaturing conditions after expressing the target polypeptide, wherein the target polypeptide is in a liquid phase if expressed in soluble form, and in a solid phase if expressed in insoluble form; and
determining the amount of soluble target polypeptide in the liquid phase.

51. The method of claim 50, wherein the target polypeptide comprises a molecular tag and the method further comprises:
removing an aliquot of the liquid phase after lysing the cells; and
contacting the target polypeptide with a detection reagent that binds to the molecular tag to determine the amount of soluble target polypeptide in the liquid phase.

52. The method of claim 51, wherein the molecular tag comprises an epitope for an antibody, a poly-histidine tag, or a FLAG™ peptide.

53. The method of claim 51, wherein the aliquot is placed on a solid support to which the target polypeptide binds prior to contacting the polypeptide with the detection reagent.

54. The method of claim 53, wherein the solid support is composed of a material selected from the group consisting of glasses, plastics, polymers, metals, metalloids, ceramics, and organics.

55. The method of claim 54, wherein the solid support comprises a microtiter plate, a nitrocellulose membrane, a nylon membrane, a derivatized nylon membrane, or an agarose particle.

56. A method of identifying mutations in a cell that alter the solubility of a target polypeptide comprising:
a) treating a cell with a mutagen;
b) introducing into the cell:
i) a solubility reporter nucleic acid that comprises a protein solubility responsive promoter operably linked to a reporter gene; and
ii) a target polypeptide-expressing nucleic acid that comprises a polynucleotide that encodes a target polypeptide;
c) culturing the cell under conditions favorable for expression of the target polypeptide;
d) measuring expression of the reporter gene; and
e) comparing the level of expression of the reporter gene in the cell with the level observed in an unmutated cell that comprises the solubility reporter nucleic acid and the target polypeptide-expressing nucleic acid to identify a cell that comprises a mutation that alters the solubility of the target polypeptide.

57. The method of claim 56, wherein the cell is treated with the mutagen after introducing either or both of the solubility reporter nucleic acid and the target polypeptide-expressing nucleic acid into the cell.

58. The method of claim 56, wherein the cell is a prokaryotic cell.

59. The method of claim 58, wherein the cell is an E. coli cell.

60. The method of claim 56, wherein the cell is a eukaryotic cell.

61. The method of claim 56, wherein the solubility is altered to enhance solubility.

62. The method of claim 56, wherein the solubility is altered to decrease solubility.

63. A method for identifying alterations to a polynucleotide that encodes a target polypeptide that alter the solubility of the target polypeptide, the method comprising:
a) altering a polynucleotide that encodes the target polypeptide to form an altered polynucleotide;
b) introducing into a cell:
i) a solubility reporter nucleic acid that comprises a protein solubility responsive promoter operably linked to a reporter gene; and
ii) a target polypeptide-expressing nucleic acid that comprises the altered polynucleotide;
c) culturing the cell under conditions favorable for expression of the target polypeptide;
d) measuring the expression of the reporter gene; and
e) comparing the level of expression of the reporter gene with the level observed in a cell with an unaltered polynucleotide that encodes the target polypeptide, to identify an alteration to the polynucleotide that changes the solubility of the encoded target polypeptide.

64. A method to identify variations in a process for biosynthesis of a target polypeptide that alter the solubility of the target polypeptide, the method comprising:
culturing a host cell under alternative conditions in which the target polypeptide is expressed, wherein the host cell comprises:
a) a solubility reporter nucleic acid that comprises a protein solubility responsive promoter operably linked to a reporter gene; and
b) a target polypeptide-expressing nucleic acid that comprises a polynucleotide that encodes a target polypeptide; and
comparing the expression of the reporter gene by host cells grown under each of the alternative conditions.

65. The method of claim 64, wherein at least two cells are cultured and the expression of the reporter gene in each cell is compared, thereby identifying a cell that expresses an altered amount of soluble target polypeptide.

66. The method of claim 64, wherein the protein solubility responsive promoter is upregulated if the target polypeptide is expressed in insoluble form, and expression of the reporter gene at a lower level is indicative of a process condition that results in greater expression of soluble target polypeptide.

67. A method of screening an expression library to identify library members that express soluble target polypeptide, the method comprising:
a) introducing a plurality of expression vectors that each comprise a polynucleotide that encodes a target polypeptide into a plurality of host cells to create an expression library, wherein the host cells comprise a solubility reporter nucleic acid that comprises a protein solubility responsive promoter operably linked to a reporter gene;
b) culturing the host cells under conditions in which the target polypeptides are expressed; and
c) detecting expression of the reporter gene, thereby identifying library members that express soluble target polypeptides.

68. The method of claim 67, wherein the protein solubility responsive promoter is upregulated when the target polypeptide is expressed in insoluble form, and host cells that express soluble target polypeptides express the reporter gene at a decreased level compared to host cells that express insoluble target polypeptides.

69. The method of claim 67, wherein the protein solubility responsive promoter is downregulated when the target polypeptide is expressed in insoluble form, and host cells that express soluble target polypeptides express the reporter gene at an increased level compared to host cells that express insoluble target polypeptides.

70. The method of claim 69, wherein the reporter gene comprises a selectable marker and host cells are grown under selective conditions, thereby selecting for host cells that express soluble target polypeptides.

71. A method of identifying an antibiotic agent, the method comprising:
contacting a cell that comprises a solubility reporter nucleic acid with a candidate antibiotic agent, wherein the solubility reporter nucleic acid comprises a protein solubility responsive promoter operably linked to a reporter gene; and
detecting the level of expression of the reporter gene, wherein a change in the expression level of the reporter gene in a cell contacted with the candidate antibiotic agent, compared to reporter gene expression level in a cell which is not contacted with the candidate antibiotic agent, is indicative of an agent that inhibits protein folding in the cell.

72. The method of claim 71, wherein the protein solubility responsive promoter comprises a polynucleotide that comprises a regulatory region of a gene listed in Table 1.

73. A method of identifying a promoter that is differentially regulated in response to expression of an insoluble polypeptide in a host cell that comprises the promoter, the method comprising:
a) providing a host cell that comprises:
i) a solubility reporter nucleic acid that comprises a putative protein solubility responsive promoter operably linked to a reporter gene; and
ii) a target polypeptide-expressing nucleic acid that comprises a polynucleotide that encodes a target polypeptide;
b) culturing the host cell under conditions in which the target polypeptide is expressed in insoluble form; and
c) determining whether expression of the reporter gene is increased or decreased, thereby determining whether the putative protein solubility responsive promoter is differentially regulated in response to expression of an insoluble polypeptide in the host cell.

74. The method of claim 73, wherein the putative protein solubility responsive promoter is a heat shock promoter.

75. The method of claim 73, wherein the putative protein solubility responsive promoter is a eukaryotic promoter.

76. The method of claim 73, wherein the putative protein solubility responsive promoter is a prokaryotic promoter.
